(19) United States
(12) Patent Application Publication
(10) Pub. No.: US 2017/0112674 A1
(43) Pub. Date: Apr. 27, 2017

(54) Dressing and Dressing Assembly for Preventing Pressure Ulcers

(71) Applicant: Hill-Rom Services, Inc., Batesville, IN (US)

(72) Inventors: David L. Ribble, Indianapolis, IN (US); Kirsten M. Emmons, Batesville, IN (US); Eric D. Agdeppa, Cincinnati, OH (US); Charles A. Lachenbruch, Batesville, IN (US); David L. Bedel, Oldenburg, IN (US)

(21) Appl. No.: 15/296,072

(22) Filed: Oct. 18, 2016

Related U.S. Application Data
(60) Provisional application No. 62/246,750, filed on Oct. 27, 2015.

(51) Int. Cl. A61F 13/02

(52) U.S. Cl. CPC A61F 13/022; A61F 13/025; A61F 13/0253; A61F 13/0256; A61F 13/0203; A61F 13/0233

(57) ABSTRACT

A dressing for guarding against the development of pressure ulcers has a recipient side and an environmental side. The dressing comprises a moisture barrier layer whose components include a sacral member, a left gluteal member and a right gluteal member. Each gluteal member has a base and an extension which extends longitudinally inferiorly further than the inferior edge of the sacral member by a dimension which is approximately no less than the longitudinal length of the sacral member. The dressing also includes a connective element on the recipient side of the dressing. The connective element is arranged to provide a zone of occlusion. In some embodiments the dressing includes electrodes and is a component of a dressing assembly which includes an exciter for stimulating gluteal muscles.

### TECHNICAL FIELD

The subject matter described herein relates to a dressing and a dressing assembly for preventing pressure ulcers throughout a portion the human posterior region extending longitudinally from approximately the sacrum to the ischeal tuberosities and laterally across the gluteal muscles.

### BACKGROUND

Individuals such as hospital patients who are immobilized in a bed for an extended time are susceptible to developing pressure ulcers. One anatomical region of special concern is the patient's posterior, particularly the region extending longitudinally from approximately the sacrum to the ischeal tuberosities (ITs) and laterally across the gluteal muscles particularly the gluteus maximus and gluteus medius. Pressure ulcers in the vicinity of the sacrum and the gluteal muscles are often the result of moisture on the skin and friction and shear acting on or experienced by the skin or the underlying tissue. Pressure ulcers associated with the ischeal tuberosities are sometimes a form of pressure ulcer referred to as a deep tissue injury (DTI). In their early stages of development deep tissue injuries might not present any external signs of their existence. As a result the presence of a deep tissue injury is often difficult to detect until the injury breaks through the skin, by which time the condition is painful, susceptible to infection, and difficult to treat successfully. Accordingly, it is desirable to develop ways of preventing the development of pressure ulcers. It is particularly desirable to develop devices that prevent pressure ulcers across a large portion of the posterior even though the preventive measures preferred for one anatomical sub-region (e.g. the sacral region) may differ from the preventive measures preferred for another anatomical sub-region (e.g. the ITs).

### SUMMARY

A dressing for guarding against the development of pressure ulcers has a recipient side and an environmental side. The dressing comprises a moisture barrier layer whose components include a sacral member, a left gluteal member and a right gluteal member. Each gluteal member has a base and an extension which extends longitudinally inferiorly further than the inferior edge of the sacral member by a dimension which is approximately no less than the longitudinal length of the sacral member. The dressing also includes a connective element on the recipient side of the dressing. The connective element is arranged to provide a zone of occlusion. In some embodiments the dressing includes electrodes and is a component of a dressing assembly which includes an exciter for stimulating gluteal muscles.

### BRIEF DESCRIPTION OF THE DRAWINGS

The foregoing and other features of the various embodiments of the dressing and dressing assembly described herein will become more apparent from the following detailed description and the accompanying drawings in which:

FIG. 1 is a plan view of a dressing as seen from the environmental side thereof with dashed lines segmenting the dressing into members useful for describing certain embodiments.

FIG. 2 is an elevation view in the direction 2-2 of FIG. 1 showing a moisture barrier layer and a connective element and also showing the dressing applied to the skin of a care recipient.

FIG. 3A is a view similar to that of FIG. 1 showing the dressing applied to a care recipient.

FIGS. 3B and 3C are views showing human anatomical features useful for describing the dressing in relation to human anatomy.

FIG. 4 is a plan view showing a variant of the dressing in which inner edges of the gluteal extensions converge more decidedly toward each other than do the inner edges of the variant of FIG. 1.

FIGS. 5-12 are a series of plan views of a dressing as seen from the recipient side thereof and a set of elevation views showing various arrangements of the connective element of the dressing.

FIGS. 13-14 are a plan view and an elevation view showing a zone of occlusion for a dressing in which the connective element is an adhesive layer which is spatially coextensive with or nearly spatially coextensive with the moisture barrier layer.

FIGS. 15-16 are a plan view and an elevation view similar to those of FIGS. 13-14 showing a zone of occlusion for a dressing in which the connective element is a strip of adhesive.

FIG. 17 is an elevation view illustrating a connective element embodied as an adhesive layer having one-way liquid absorbancy.

FIG. 18 is an elevation view illustrating a connective element embodied as an adhesive layer having one-way liquid permeability.

FIGS. 19-30 are a series of plan views of a dressing as seen from the recipient side thereof and a set of elevation views showing various arrangements of the connective element of the dressing with the connective element broken away to expose an auxiliary layer embodied as a moisture management layer, specifically a liquid absorbant layer.

FIGS. 31-42 are a series of plan views of a dressing as seen from the recipient side thereof and a set of elevation views showing various arrangements of the connective element of the dressing with the connective element broken away to expose an auxiliary layer embodied as a moisture management layer, specifically a liquid permeable layer.

FIGS. 43-50 are a series of plan views of a dressing as seen from the recipient side thereof and a set of elevation views showing various arrangements of the connective element of the dressing with the connective element broken away to expose an auxiliary layer embodied as a pressure equalizing layer.

FIGS. 51A-51C are elevation views illustrating the functioning of the pressure equalizing layer.

FIG. 51D is an elevation view showing a dressing with a pressure equalization zone whose thickness exceeds the baseline thickness of the dressing outside the planform of the pressure equalization zone and in which the local increase in thickness is attributable to the thickness of the auxiliary layer.

FIG. 51E is an elevation view showing a dressing with a pressure equalization zone whose thickness exceeds the baseline thickness of the dressing outside the planform of the pressure equalization zone and in which the local increase in thickness is attributable to a local increase in thickness of moisture barrier layer.

FIG. 51F is an elevation view of a dressing with a pressure equalizing zone having a customizable thickness attributable to multiple peel-away sublayers.

FIG. 51G is an illustration of a stockpile of classified dressings in which the thickness of the pressure equalizing zone in a given class differs from the thickness of zone P_{ZE} in the other classes.

FIGS. 52-59 are a series of plan views of a dressing as seen from the recipient side thereof and a set of elevation views showing various arrangements of the connective element of the dressing and also showing an auxiliary layer embodied as a pressure equalizing layer in conjunction with a moisture management layer.

FIGS. 60-64 are views illustrating example variations on the planform of the dressing.

FIG. 65 is a view similar to those of FIGS. 60-64 showing a dressing in which a sacral member, a left gluteal member, and a right gluteal member are individual members rather than integrated into a single unit.

FIGS. 66-67 are a side elevation view and a posterior view illustrating threat-susceptible regions of a target anatomy in connection with a description of criteria for sizing and proportioning the dressing in relation to features of human anatomy.

FIGS. 68-69 are views similar to the view of FIG. 3C showing criteria for sizing and proportioning the dressing in relation to features of human anatomy.

FIG. 70 shows a plan view of a preventive dressing assembly which comprises a dressing having an array of four electrodes and in which the dressing assembly also comprises an exciter for exciting the electrodes.

FIG. 71 is a chart showing example excitation patterns for the electrodes of the dressing assembly of FIG. 70 and also enumerating the gluteal muscle or muscles stimulated by each sample excitation pattern.

FIG. 72 shows a plan view of a preventive dressing assembly which comprises a dressing having an array of six electrodes and in which the dressing assembly also comprises an exciter for exciting the electrodes.

FIG. 73 is a chart showing example excitation patterns for the electrodes of the dressing assembly of FIG. 72 and also enumerating the gluteal muscle or muscles stimulated by each sample excitation pattern.

FIGS. 74-75 are a plan view and an elevation view of a dressing having a nonocclusive external layer and a nonocclusive connective element embodied as a distributed adhesive, and showing fluid ingress through the external layer into a zone of coverage and fluid ingress into the connective element and into the zone of coverage.

FIGS. 76-77 are a plan view and an elevation view of an embodiment similar to that of FIGS. 74-75 but in which the connective element is embodied as an adhesive strip.

FIGS. 78-79 are a plan view and an elevation view of an embodiment similar to that of FIGS. 76-77 but in which the connective element 120 is embodied as an adhesive strip 190 which blocks moisture transfer laterally and longitudinally into zone Z_{C}.

### DETAILED DESCRIPTION

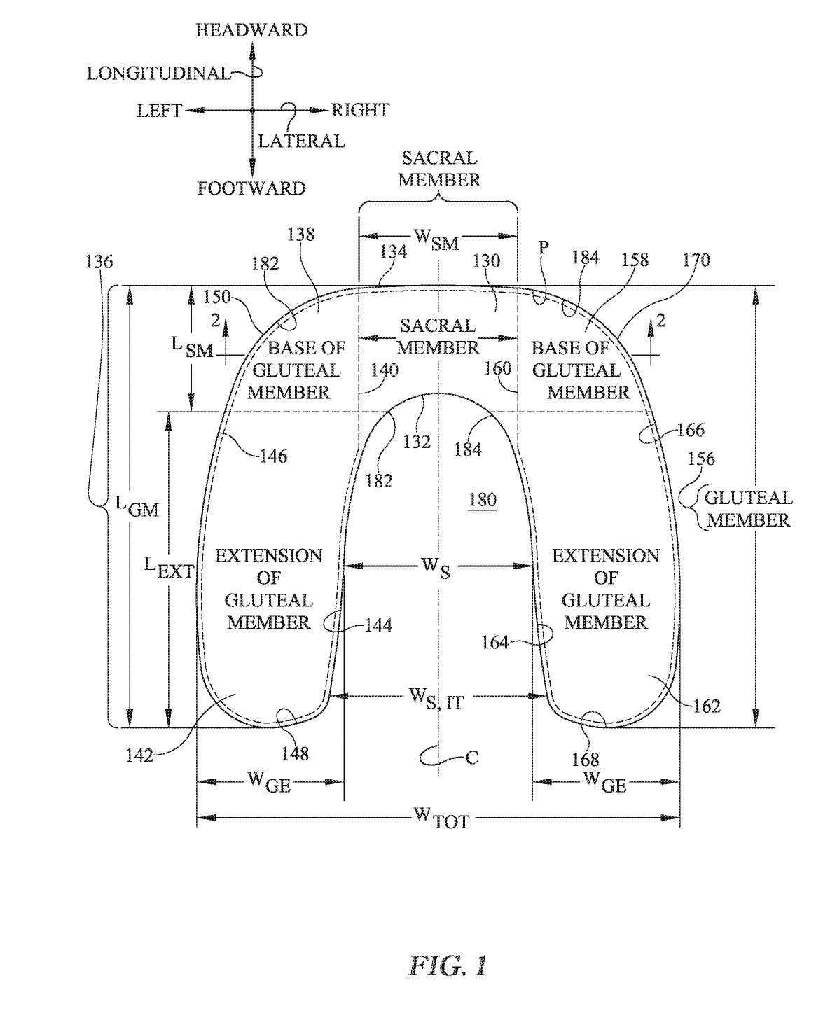

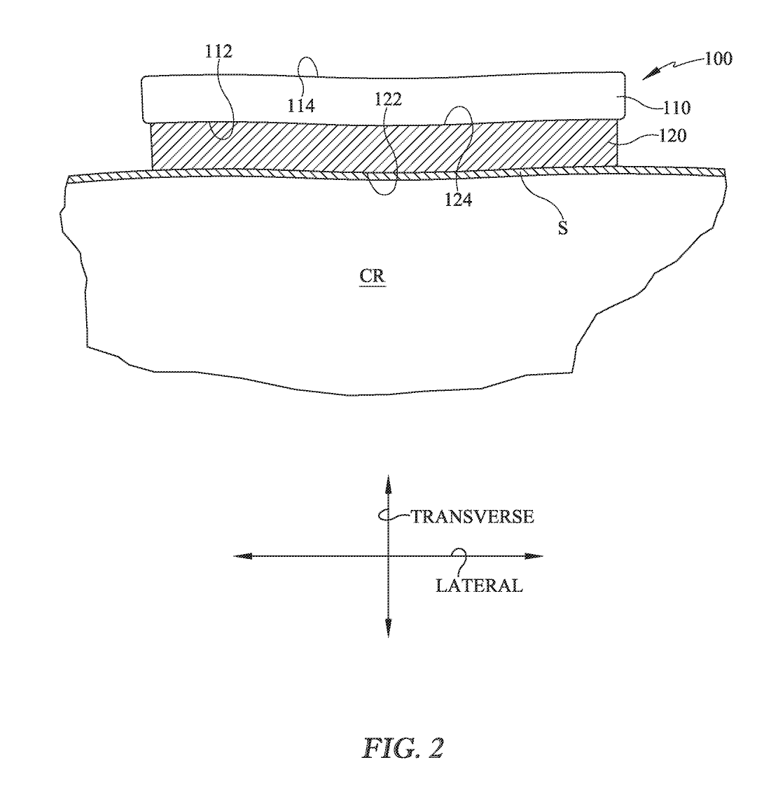

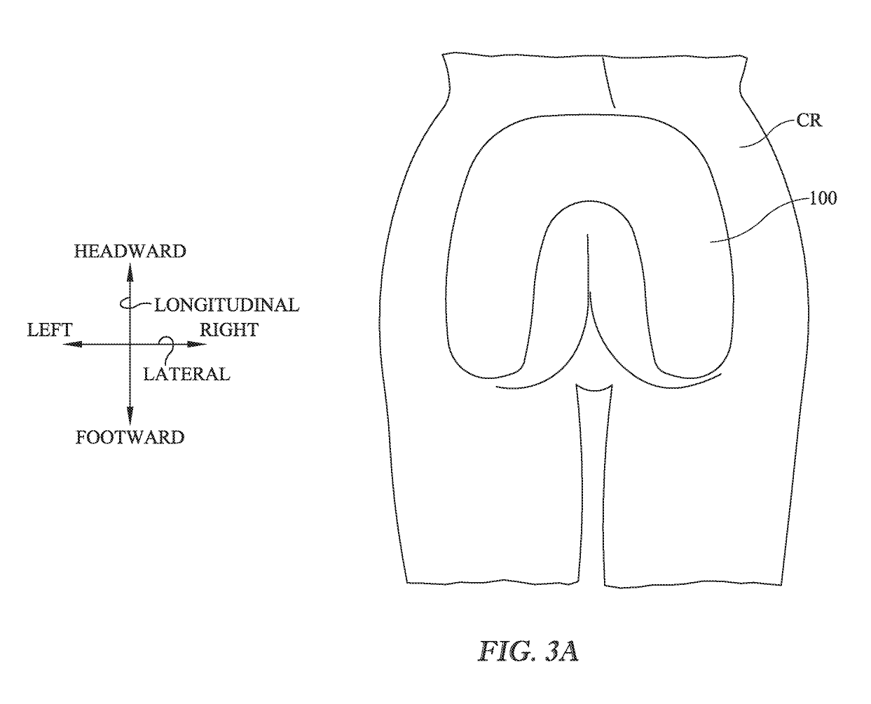

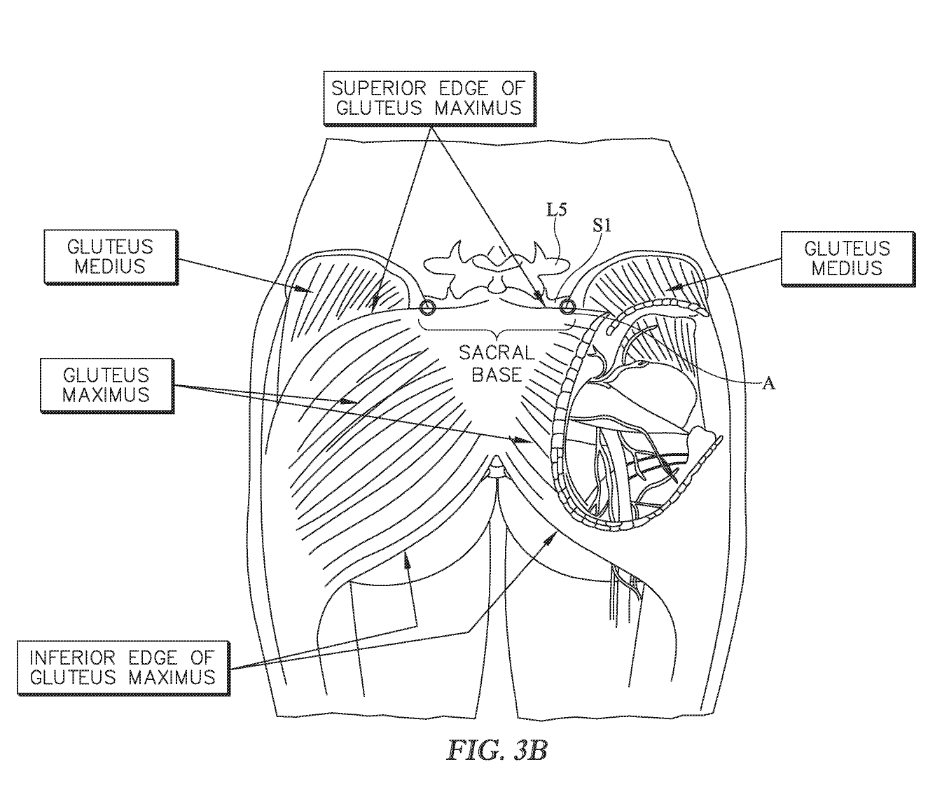

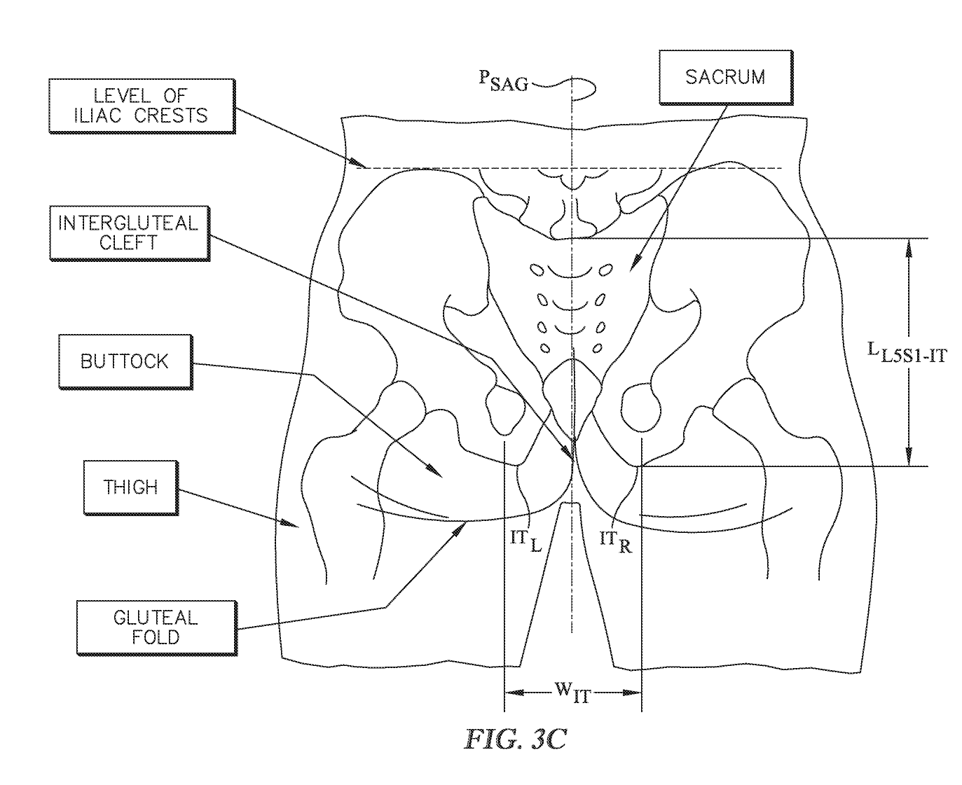

FIGS. 1-2 are schematic illustrations of an example preventive dressing 100 for a care recipient CR such as a hospital patient. FIG. 3A shows the dressing applied to the care recipient. FIGS. 3B and 3C show anatomical features useful for describing the dressing in relation to human anatomy and for understanding its operation. Selected illustrations also include reference axes indicating longitudinal, lateral, and transverse directions. When describing the dressing, the thickness of the dressing or its components is the dimension in the transverse direction. Throughout this description features similar to or the same as features previously described may be identified by the same reference numerals. Features which are specific examples of more general features may also all be identified by the same or similar reference numerals.

The dressing is configured to cover a portion of a human body, specifically a posterior region extending longitudinally from approximately the sacrum to the ischeal tuberosities (ITs) and laterally across the gluteal muscles. The dressing is in the form of a thin sheet having sufficient flexibility that it can conform to anatomical contours. In the interest of clarity the thickness of the dressing may be exaggerated in the accompanying illustrations.

The dressing comprises a moisture barrier layer 110 having a recipient side 112 intended to face toward the care recipient and an environmental side 114 which is the side intended to face away from the care recipient. The environmental side is exposed to the external environment. The dressing also has a connective element 120 on the recipient side of the moisture barrier layer. The connective element has a recipient side 122 facing toward the care recipient and an environmental side 124 facing away from the care recipient but not necessarily exposed to the environment. The connective element is intended to hold the dressing in contact with the care recipient and is shown with its recipient side 122 in contact with the care recipient's skin S. One example of a connective element is an adhesive which adheres well to human skin but can be removed from the skin without undue difficulty or undue discomfort to the care recipient.

The dressing also has a perimeter P and a longitudinally extending centerline C. As seen in the plan view of FIG. 1 the dressing has a sacral member 130 corresponding approximately to the sacrum of a care recipient having a target anatomy, for example the anatomy of a 50th percentile United States male. The sacral member has a longitudinal length L_{SM}, a lateral width W_{SM}, an inferior edge 132 and a superior edge 134. Superior edge 134 of the sacral member may also be considered to be the superior edge of the dressing as a whole. Width W_{SM} is approximately the width of the sacral base of the target care recipient. Accordingly, sacral member 130 extends laterally leftwardly to no more than about the location “A” (FIG. 3B) of the superior edge of the gluteus maximus muscle. The dressing also includes a left gluteal member 136 having a base 138 whose right edge 140 borders the left side of the sacral member, and a left extension 142 having a laterally inner edge 144, a laterally outer edge 146 and a terminus or inferior edge 148. Collectively, inner edge 144 outer edge 146 and terminus or inferior edge 148 define a margin of the left extension. The left extension extends longitudinally inferiorly of the inferior edge 132 of the sacral member by extension dimension L_{EXT}. L_{EXT} is approximately no less than the longitudinal length L_{SM} of the sacral member. The dressing also includes a right gluteal member 156 having a base 158 whose left edge 160 borders the right side of the sacral member, and a right extension 162 having a laterally inner edge 164, a laterally outer edge 166, and a terminus or inferior edge 168. Collectively, inner edge 164, outer edge 166 and terminus or inferior edge 168 define a margin of the right extension. Taken together extension inferior edges 148, 168 define an inferior edge of the dressing as a whole. The right extension extends longitudinally inferiorly of the inferior edge 132 of the sacral member by the dimension L_{EXT}. The overall longitudinal length of the gluteal members, and therefore of the dressing as a whole, is L_{GM} which is approximately equal to the sum of L_{SM} plus L_{EXT}.

The gluteal extensions each have a width W_{GE}. The extensions define an intermember space 180 having a width W_{S} measured about midway between the inferior edges 148, 168 of the extensions and the inferior edge 132 of the sacral member. The dressing has a total width of W_{TOT} which is W_{S} plus twice W_{GE}.

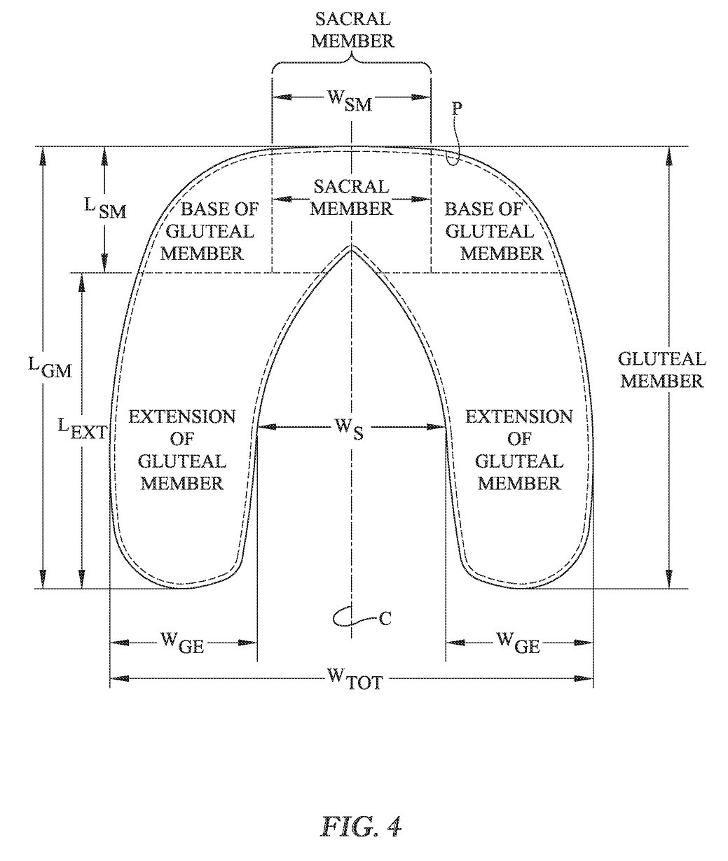

In the dressing of FIG. 1 inner edges 144, 164 of the gluteal extensions and the inferior edge 132 of the sacral member are approximately linear. Curved edge segments 182, 184 connect inferior edge 132 of the sacral member to the inner edges 144, 164 of the extensions. In another embodiment shown in FIG. 4, the inner edges converge more decidedly toward each other.

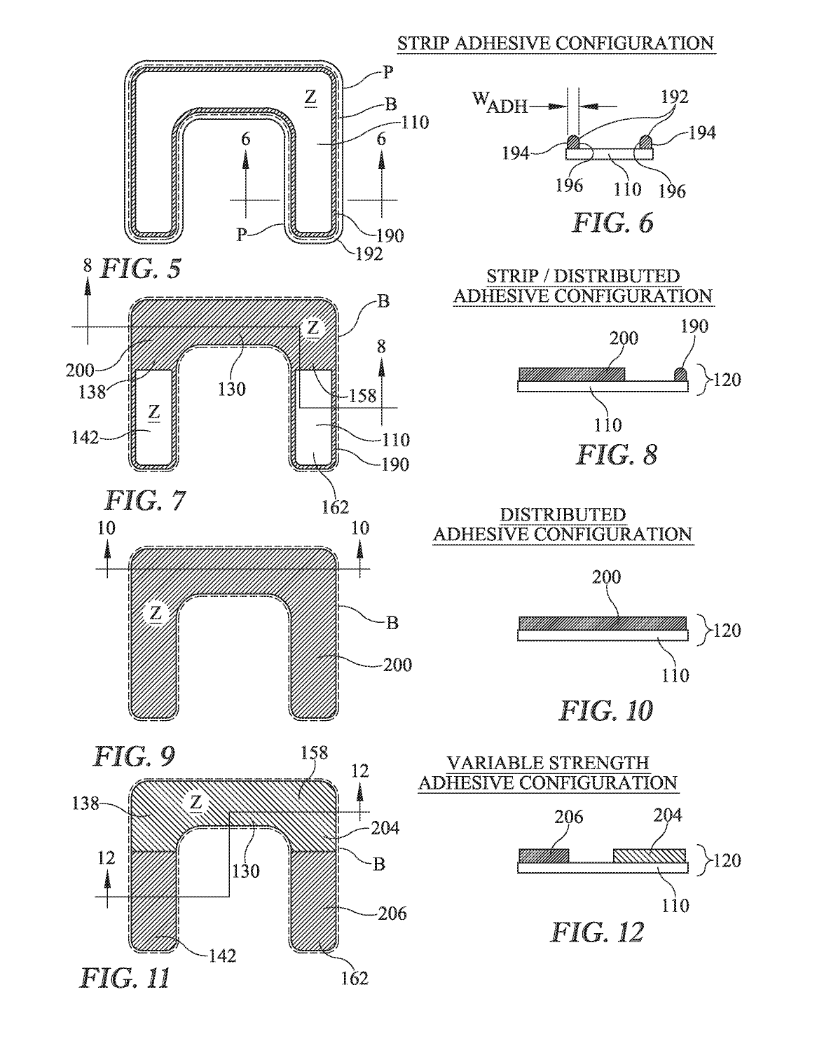

FIGS. 5-12 show various arrangements of connective element 120. In FIGS. 5-6 the connective element is a continuous strip of adhesive 190 extending along substantially the perimeter of the dressing, i.e. at perimeter P or near the perimeter but slightly offset therefrom to define a nonadhesive border 192 of the moisture barrier layer. The adhesive strip has an outer edge 194, which is the edge closer to dressing perimeter P, and an inner edge 196, which is the edge further away from the dressing perimeter. The configuration of FIGS. 5-6 is referred to as the strip adhesive configuration. In FIGS. 7-8 the connective element comprises a sacral adhesive layer 200 distributed over substantially the entirety of the recipient side of the sacral member 130 and over substantially the entirety of the gluteal bases 138, 158. The connective element of FIGS. 7-8 further comprises a continuous strip of adhesive 190 extending along substantially the margins of the gluteal member extensions 142, 162. The arrangement of FIGS. 7-8 is referred to as the strip/distributed adhesive configuration. In FIGS. 9-10 the connective element comprises an adhesive 200 which forms substantially all of the recipient side of the dressing. In other words the recipient side of the dressing is the recipient side of the adhesive. The arrangement of FIGS. 9-10 is referred to as the distributed adhesive configuration.

As seen in FIGS. 11-12, the strength of the adhesive need not be spatially uniform as indicated by the density of the crosshatching. In FIGS. 11-12 the connective element comprises a first adhesive composition 204 forming substantially all of the recipient sides of sacral member 130 and gluteal bases 138, 158, and a second adhesive composition 206 forming substantially all of the recipient side of gluteal extensions 142, 162. The first adhesive composition adheres relatively more strongly to human skin and the second adhesive composition adheres relatively more weakly to human skin. The configuration of FIGS. 11-12 is referred to as the variable strength adhesive configuration.

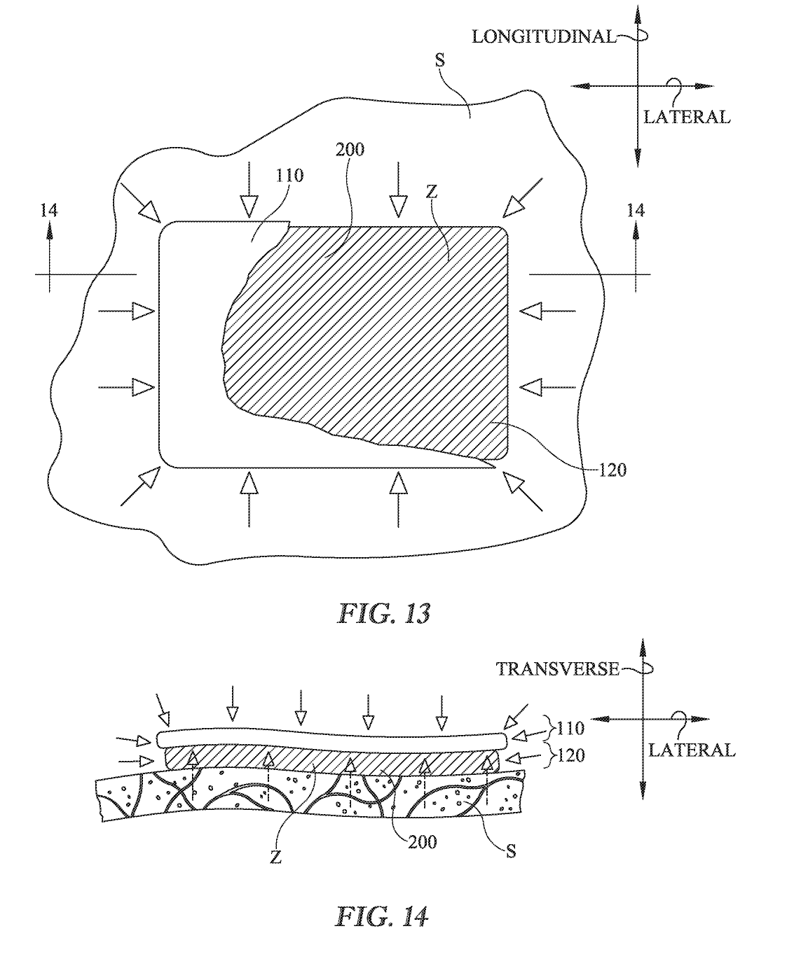

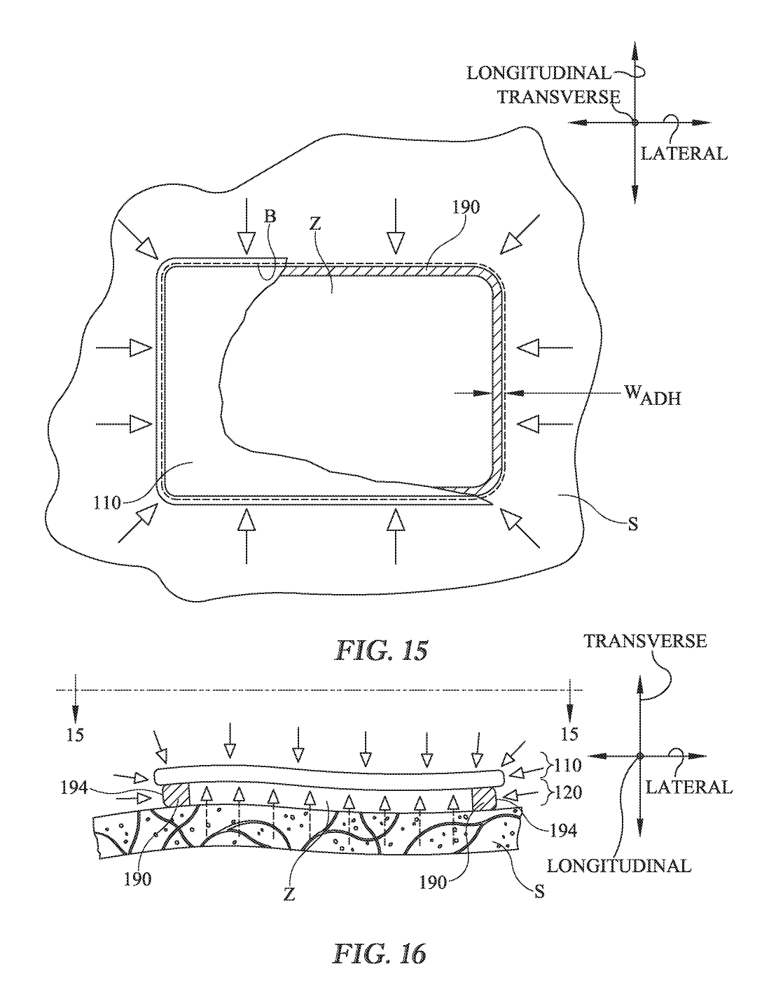

The adhesive or other connective element is arranged to provide a zone of occlusion Z. As used herein a zone of occlusion is a zone protected from environmental moisture ingress by the adhesive and the moisture barrier layer. Such protection is desirable because moisture on the skin is a factor that can lead to development of pressure ulcers. Examples of environmental moisture include liquids originating outside the zone of occlusion (e.g. accidentally spilled water, or urine from an incontinent patient). Examples of environmental moisture also include the care recipient's perspiration through skin not covered by the zone of occlusion. The protection against environmental moisture ingress is illustrated schematically in FIGS. 13-14 and 15-16 where the moisture barrier layer 110 protects against environmental moisture ingress principally in the transverse (thickness) direction while the adhesive protects against environmental moisture ingress in the lateral and longitudinal directions (solid arrows which terminate outside the occlusion zone). In the arrangement shown schematically in FIGS. 13-14 the adhesive layer 120 is spatially coextensive with or nearly spatially coextensive with the moisture barrier layer 110. The zone of occlusion is defined by the spatial extent of the adhesive. In the arrangement shown schematically in FIGS. 15-16 the adhesive layer is a strip of adhesive 190 which forms a closed boundary defined by dashed borderline B. (Borderline B in FIG. 15 and in other illustrations coincides with outer edge 194 of the adhesive strip but is shown slightly offset edge 194 to preserve the visibility of both the edge and the borderline.) The zone of occlusion is defined by the adhesive strip and includes the region within the closed boundary. In other words the zone of occlusion includes the width W_{ADH} of the adhesive strip.

Moisture in the form of the care recipient's perspiration through the skin covered by the zone of occlusion (dashed arrows) of FIGS. 14 and 16 is not environmental moisture. Therefore the status of zone Z as a zone of occlusion in FIGS. 13-16 is not defeated by such perspiration.

Returning momentarily to FIGS. 5, 7, 9 and 11, dashed borderline B shows the zones of occlusion Z for each of the described adhesive configurations. In FIG. 5 zone Z is the zone enclosed by outer edge 194 of adhesive strip 190 (or equivalently borderline B) In FIGS. 7, 9 and 11 zone Z is the planform of the entire dressing because the spatially distributed adhesive 200 extends to dressing perimeter P (FIGS. 9, 11) and because outer edge 194 of adhesive strip 190 (FIG. 7) is exactly at perimeter P.

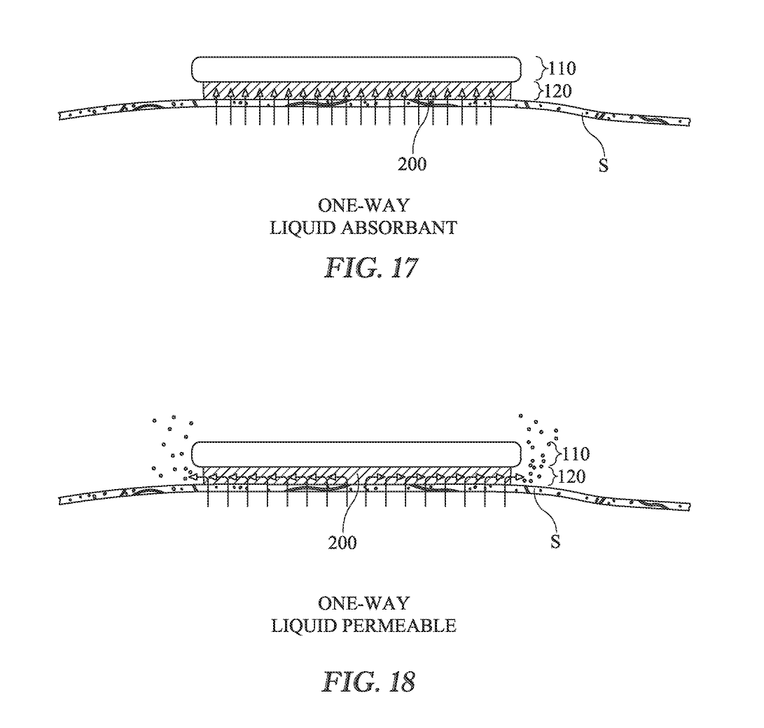

If the adhesive or other connective element is a strip as in FIGS. 5-6, or as shown along the margins of the gluteal extensions in FIGS. 7-8, the adhesive may or may not be one-way liquid absorbant. However in the examples of this specification the strip adhesive is always considered to be one-way liquid absorbant. If the adhesive is distributed as in the remainder of FIGS. 7-8 or as in FIGS. 9-10, the adhesive is one-way liquid absorbant. As shown schematically in FIG. 17 the direction of the one-way absorbancy is the direction that draws moisture, such as perspiration, away from the care recipient's skin S thereby eliminating a risk factor (excessive moisture on the skin) for pressure ulcer development. Alternatively, as seen in FIG. 18, the adhesive may be one-way liquid permeable. The direction of the one-way permeability is the direction that transfers moisture away from the care recipient's skin. The one-way liquid permeability not only draws moisture away from the care recipient's skin (similar to the liquid absorbant layer of FIG. 17) but also discharges the moisture to the environment where it can evaporate or be actively disposed of.

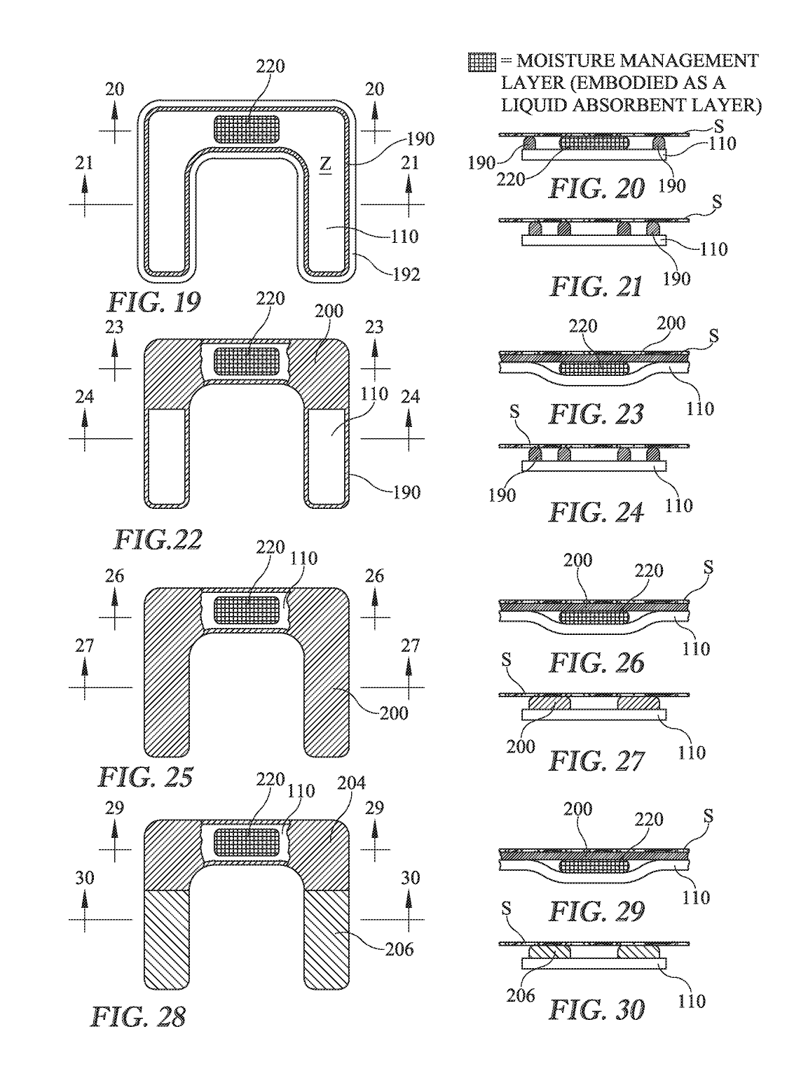

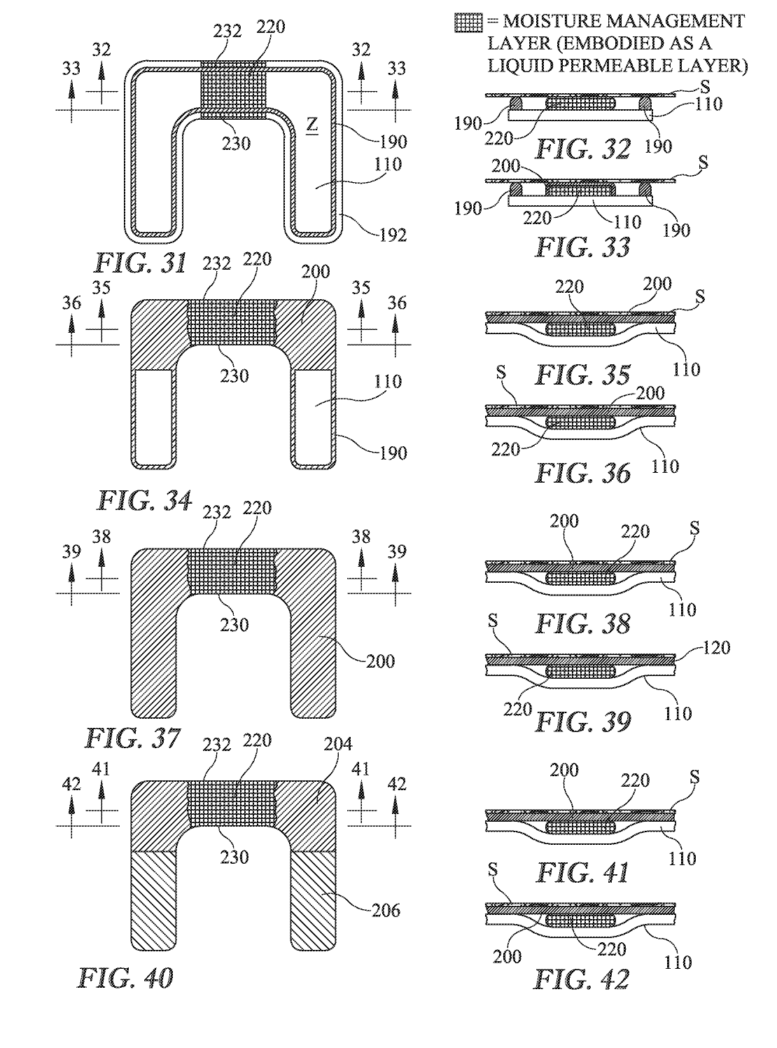

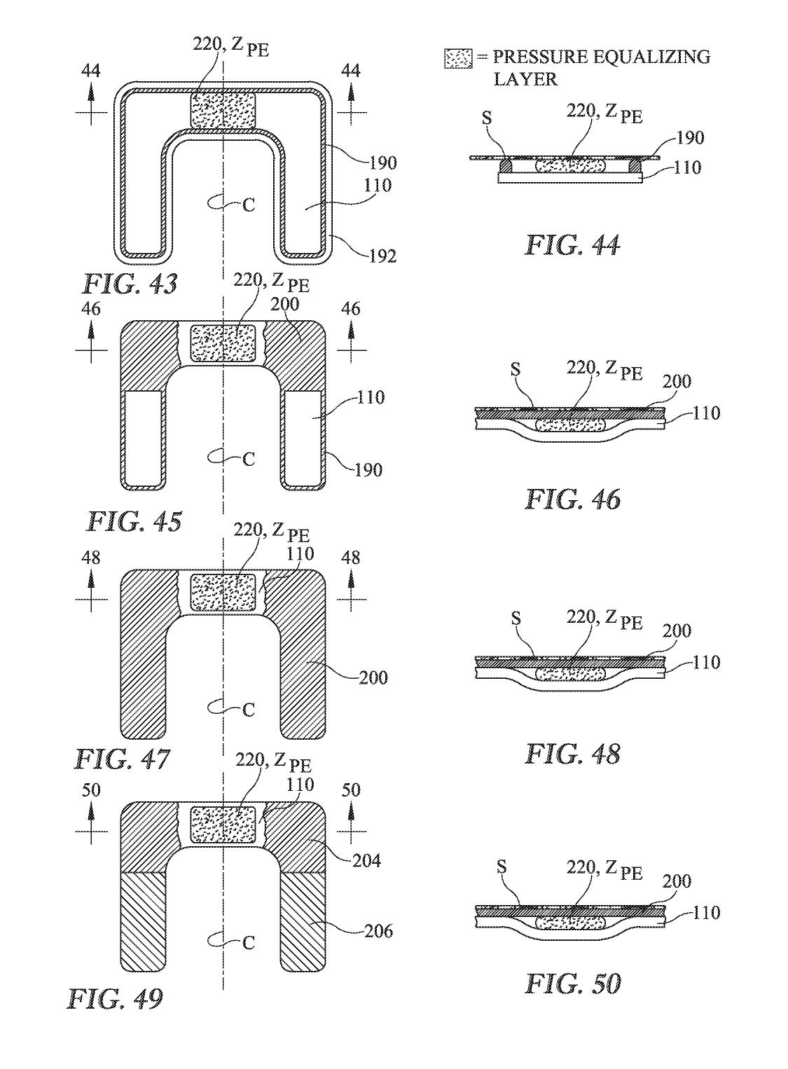

FIGS. 19-50 and 52-59 are similar to FIGS. 5-12 but show an auxiliary layer 220 in addition to the moisture barrier layer 110 and the adhesive layer (embodied as a spatially distributed layer 200 or as an adhesive strip 190). The auxiliary layer comprises a liquid absorbant layer, a liquid permeable layer, a pressure equalizing layer or some combination of such layers. This specification employs the phrase “moisture management layer” to mean the liquid absorbant layer when used alone, the liquid permeable layer when used alone, or the liquid absorbant layer and liquid permeable layer used together.

FIGS. 19-30 are similar to FIGS. 5-12 but show the auxiliary layer 220, embodied as a moisture management layer, specifically a one-way liquid absorbant layer. The direction of the one-way absorbancy is the direction that transfers moisture, such as perspiration, away from the care recipient's skin S, not toward the care recipient's skin (FIGS. 19-21) or that transfers moisture away from the adhesive (FIGS. 22-30) similar to the one-way liquid absorbancy of adhesive 200 of FIG. 17. FIGS. 19-21 show the liquid absorbant layer 220 used in the strip adhesive configuration (e.g. the configuration of FIGS. 5-6). In FIGS. 19-21 the liquid absorbant moisture management layer resides in the zone of occlusion. When applied to a care recipient the moisture management layer is in direct contact with the care recipient's skin S. The illustrated moisture management layer is confined to the sacral member, but could be present elsewhere in the occlusion zone instead of or in addition to being present in the sacral member. In the limit the moisture management layer is present throughout the zone of occlusion but not where it would intervene between adhesive strip 190 and the care recipient's skin. The liquid absorbant moisture management layer draws moisture away from the care recipient's skin and continues to do so until it reaches saturation. FIGS. 22-24 show liquid absorbant layer 220 used in the strip/distributed adhesive configuration (e.g. the configuration of FIGS. 7-8). FIGS. 25-27 show the liquid absorbant layer 220 used in a distributed adhesive configuration (e.g. the configuration of FIGS. 10-11). FIGS. 28-30 show the liquid absorbant layer 220 used in the variable strength adhesive configuration (e.g. the configuration of FIGS. 11-12). In the arrangements of FIGS. 19-30, the moisture management 220 layer resides in the zone of occlusion and is transversely between moisture barrier layer 110 and the adhesive or other connective element 120 (which is illustrated as a distributed adhesive 200 in FIGS. 19-30).

FIGS. 31-42 are similar to FIGS. 5-12 but show the moisture management layer embodied as a one-way liquid permeable layer 220. The direction of the one-way permeability is the direction that transports moisture, such as perspiration, away from the care recipient's skin S, not toward the care recipient's skin (FIGS. 31-33) or that transports moisture away from the adhesive (FIGS. 34-42) similar to the one-way liquid permeability of adhesive 200 of FIG. 18. A noteworthy difference between the liquid permeable layer of FIGS. 31-42 and the liquid absorbant layer of FIGS. 19-30 is that the liquid permeable layer is in communication with or otherwise exposed to the external environment. In FIGS. 31-42 the liquid permeable layer is exposed to the environment by virtue of extending longitudinally to inferior edge 132 and superior edge 134 of sacral member 130. Because inferior and superior edges 230, 232 of the liquid permeable layer are exposed to the environment, liquid absorbed by the layer can be discharged to the environment. However because the one-way permeability of the liquid permeable layer is in a direction that transports liquid away from the zone of occlusion, the fact that edges 230, 232 are exposed to the environment does not compromise the occlusive nature of the zone of occlusion.

FIGS. 31-33 show liquid permeable layer 220 used in the strip adhesive configuration (e.g. the configuration of FIGS. 5-6). In FIGS. 31-33 the liquid permeable moisture management layer resides in the zone of occlusion. When applied to a care recipient the moisture management layer is in direct contact with the care recipient's skin S. The illustrated moisture management layer is confined to the sacral member, but could be present elsewhere in the occlusion zone instead of or in addition to being present in the sacral member. In the limit the moisture management layer is present throughout the zone of occlusion but not where it would intervene between adhesive strip 190 and the care recipient's skin. The liquid permeable moisture management layer draws moisture away from the care recipient's skin and discharges it to the environment. FIGS. 34-36 show the moisture permeable layer 220 used in the strip/distributed adhesive configuration (e.g. the configuration of FIGS. 7-8). FIGS. 37-39 show the moisture permeable layer 220 used in a distributed adhesive configuration (e.g. the configuration of FIGS. 10-11). FIGS. 40-42 show the moisture permeable layer 220 used in the variable strength adhesive configuration (e.g. the configuration of FIGS. 11-12). In the arrangements of FIGS. 31-42, the moisture management layer resides in the zone of occlusion and is transversely between moisture barrier layer 110 and the adhesive or other connective element 120 which is illustrated as a distributed adhesive 200 in FIGS. 31-42.

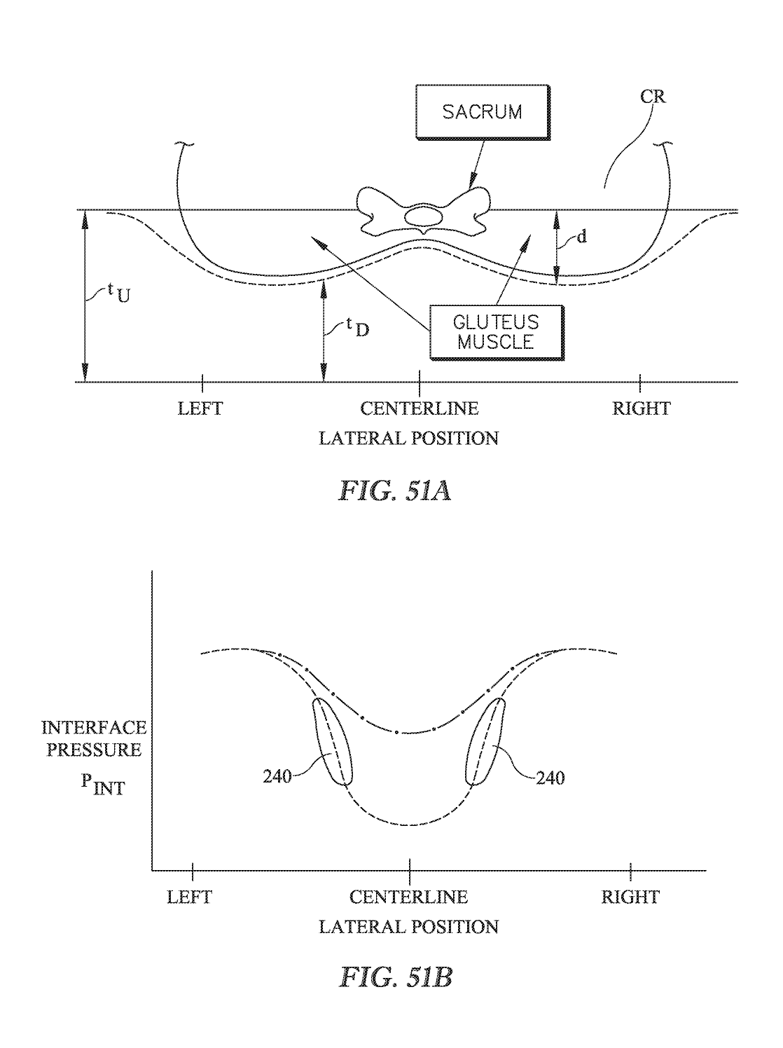

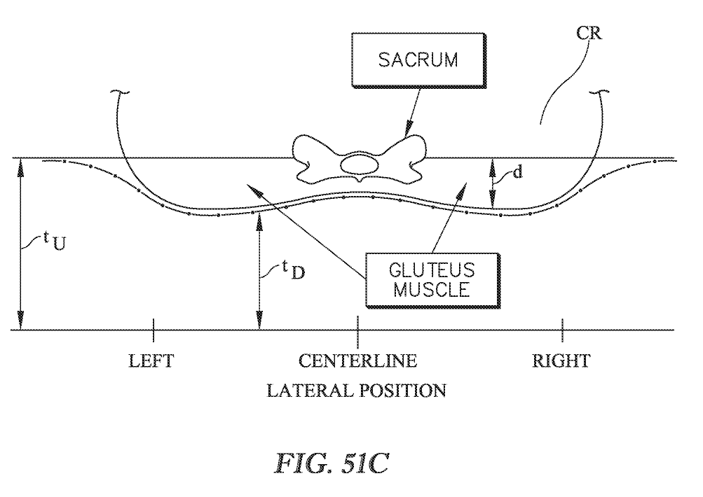

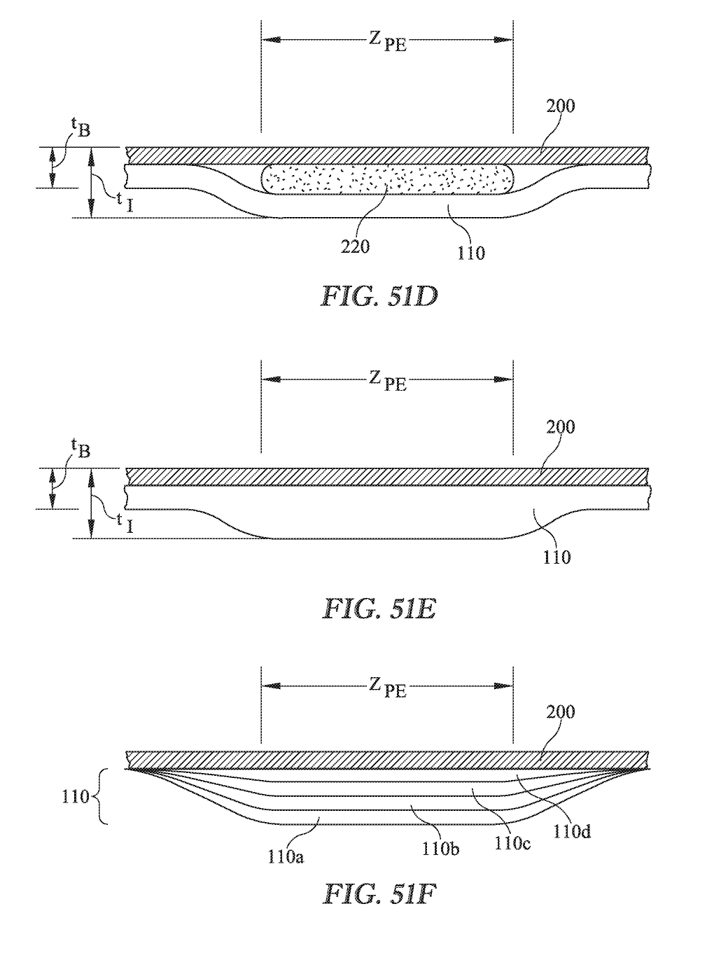

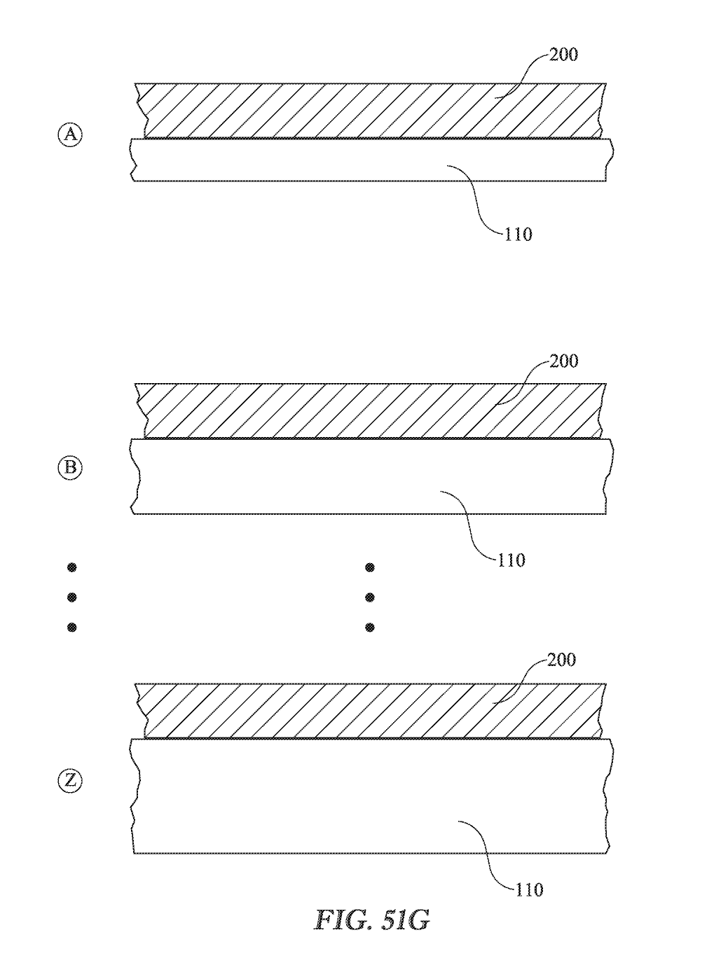

FIGS. 43-50 are similar to FIGS. 5-12 but show the auxiliary layer embodied as a pressure equalizing layer 220. FIGS. 51A-51C illustrate the function of the pressure equalizing layer. Referring first to FIG. 51A, a mattress has an undeflected thickness t_{U}, which is substantially uniform in the lateral direction. The weight of the care recipient CR deforms the mattress to a laterally nonuniform deflected thickness t_{D} as indicated by the dashed line profile. The deflection d is relatively small in the vicinity of the care recipient's sacrum and larger along the gluteal muscles. Referring additionally to the dashed line of FIG. 51B, the corresponding interface pressure P_{INT} is therefore smaller at the sacrum and larger along the gluteal muscle. The pressure gradients at the steep portions 240 of FIG. 51B correspond to a region of high shear both at the skin and transversely through the muscle tissue. Referring to FIGS. 43-50 and 51C, the pressure equalizing layer 220 is a layer of material which spans laterally across centerline C of the dressing in the zone of occlusion to define a pressure equalizing zone Z_{PE}. The thickness and/or stiffness of the pressure equalizing material layer causes the pressure equalizing zone to exhibit a stiffness (i.e. a resistance to deflection) which is greater than the stiffness of the dressing laterally to the left and right of the pressure equalizing zone. As seen in FIG. 51C compared to FIG. 51A, this causes thickness t_{D} and deflection d to be more laterally uniform. As a result the lateral gradient of interface pressure is reduced (FIG. 51B dot-dashed profile versus the dashed profile) and therefore so is shear.

As described above the thickness of the pressure equalizing layer may cause the pressure equalizing zone to exhibit an increased stiffness in comparison to regions of the dressing outside the planform of the pressure equalizing layer and pressure equalizing zone. However an increased thickness of the pressure equalizing layer, relative to the thickness of the dressing outside the pressure equalizing zone/layer, may be sufficient to create a pressure equalizing zone even if the increased thickness is not accompanied by an increase in stiffness. In that case the increased thickness of the pressure equalizing layer acts on its own to yield the pressure equalization of FIG. 51B, although the increased thickness may diminish patient comfort. FIGS. 51D and 51E each show a dressing with a pressure equalization zone whose thickness t_{I} exceeds the baseline thickness t_{B} of the dressing outside the planform of the pressure equalization zone. In FIG. 51D the local increase in thickness is attributable to the thickness of auxiliary layer 220. In FIG. 51E the local increase in thickness is attributable to a local increase in thickness of moisture barrier layer 110.

FIG. 51F shows a dressing with a pressure equalizing zone having a customizable thickness. The customizable thickness is the result of moisture barrier layer 110 being comprised of multiple peel-away sublayers, for example 110a, 110b, 110c, 110d such that sublayer 110a can be peeled off sublayer 110b, sublayer 110b can be peeled off sublayer 110c and sublayer 110c can be peeled off sublayer 110d. In practice a caregiver may leave all the sublayers in place or may peel away one or more sublayers. As successive sublayers are removed the pressure equalizing property of the pressure equalizing zone is diminished.

FIG. 51G shows a variation on the theme of FIG. 51F. FIG. 51G shows a stockpile of classified dressings, for example classes A through Z. Zone P_{ZE} is a classified zone having a class specific thickness. The thickness of zone P_{ZE} in a given class differs from the thickness of zone P_{ZE} in the other classes. A manufacturer would produce dressings in each of two or more classes each of which corresponds to a class specific thickness. In practice the caregiver selects a dressing from the class he believes is best suited for the patient under his care.

In one embodiment the pressure equalizing zone is confined to the sacral member, i.e. is confined to the immediate vicinity of the care recipient's sacrum, and does not extend laterally to edges 140, 160 of gluteal bases 138, 158. In another embodiment the pressure equalizing zone extends laterally to the edges 140, 160 of the gluteal bases. Either way the stiffness of the pressure equalizing zone is greater than the stiffness of the left gluteal base 138 and is also greater than the stiffness of the right gluteal base 158. In yet another embodiment the pressure equalizing zone may extend beyond edges 140, 160 and into the gluteal bases. In addition, the dash-dot profile of FIG. 51B demonstrates that the phrase “pressure equalizing” does not mean that the pressure equalizing layer must cause the interface pressure to be substantially uniform in the lateral direction, only that it attenuate the lateral pressure gradients.

FIGS. 43-44 show pressure equalizing layer 220 used in the strip adhesive configuration of the dressing (e.g. the configuration of FIGS. 5-6). FIGS. 45-46 show the pressure equalizing layer 220 used in the strip/distributed adhesive configuration of the dressing (e.g. the configuration of FIGS. 7-8). FIGS. 47-48 show the pressure equalizing layer 220 used in the distributed adhesive configuration (e.g. the configuration of FIGS. 10-11). FIGS. 49-50 show the pressure equalizing layer 220 used in the variable strength adhesive configuration (e.g. the configuration of FIGS. 11-12). In the arrangements of FIGS. 45-50 the pressure equalizing layer is transversely between moisture barrier layer 110 and the adhesive or other connective element 120.

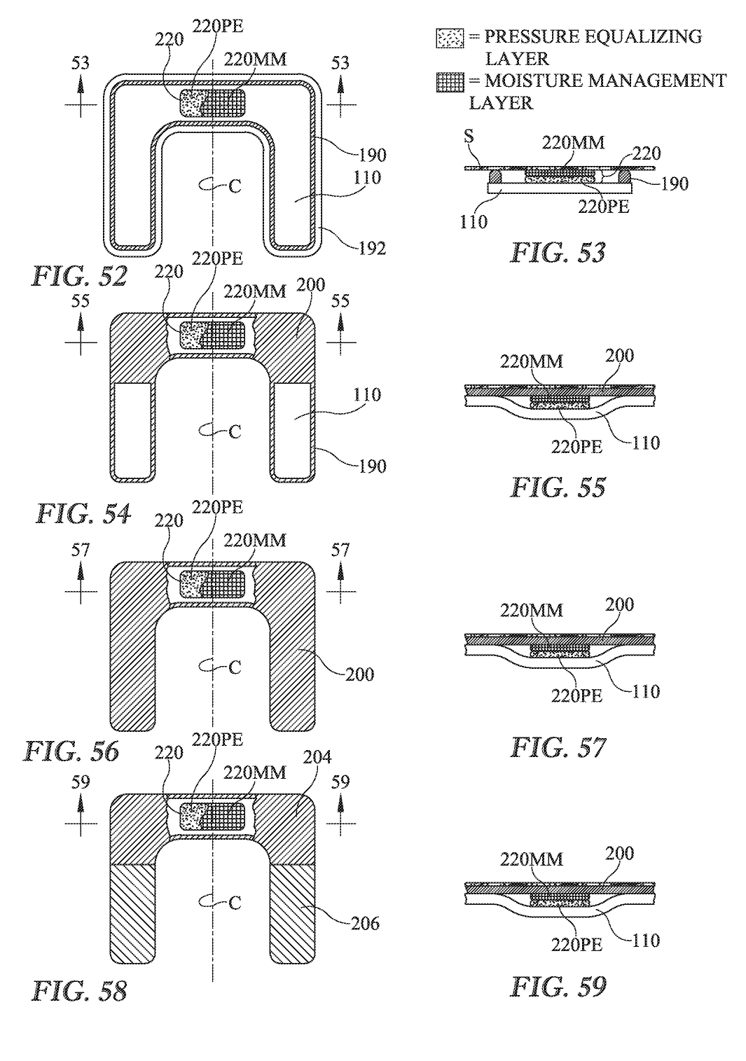

FIGS. 52-59 are similar to FIGS. 5-12 but show the auxiliary layer 220 embodied as a pressure equalizing layer 220PE in conjunction with a moisture management layer 220MM. The moisture management layer may be a liquid absorbant layer as previously described, a liquid permeable layer as previously described, or both. As seen in FIGS. 53, 55, 57 and 59 moisture management layer 220MM is on the recipient side of the pressure equalizing layer so that when the dressing is applied to a care recipient the moisture management layer is in direct contact with the care recipient's skin S or is closer to the skin than is the pressure equalizing layer. The pressure equalizing layer 220PE spans laterally across centerline C of the dressing in the zone of occlusion to define a pressure equalizing zone. In one embodiment the pressure equalizing zone is confined to the sacral member, i.e. is confined to the immediate vicinity of the care recipient's sacrum, and does not extend laterally to edges 140, 160 of gluteal bases 138, 158. In another embodiment the pressure equalizing zone extends laterally to the edges 140, 160 of the gluteal bases. Either way the stiffness of the pressure equalizing zone is greater than the stiffness of the left gluteal base 138 and is also greater than the stiffness of the right gluteal base 158. In yet another embodiment the pressure equalizing zone may extend beyond edges 140, 160 and into the gluteal bases. The illustrated moisture management layer 220 is confined to the planform of the sacral member, but could be present elsewhere in the occlusion zone, instead of or in addition to being present in the sacral member. In the limit the moisture management layer is present throughout the zone of occlusion.

FIGS. 52-53 show the combined moisture management and pressure equalizing layer 220 used in the strip adhesive configuration (e.g. the configuration of FIGS. 5-6). FIGS. 54-55 show the combined moisture management and pressure equalizing layer 220 used in the strip/distributed adhesive configuration (e.g. the configuration of FIGS. 7-8). FIGS. 56-57 show the moisture management and pressure equalizing layer 220 used in a distributed adhesive configuration (e.g. the configuration of FIGS. 10-11). FIGS. 58-59 show the moisture management and pressure equalizing layer 220 used in the variable strength adhesive configuration (e.g. the configuration of FIGS. 11-12). In the arrangements of FIGS. 54-59, the moisture management layer and the pressure equalizing layer both reside in the zone of occlusion and are both transversely between the moisture barrier layer and the adhesive layer or other connective element 120 which is illustrated as a distributed adhesive 200 in FIGS. 52-59.

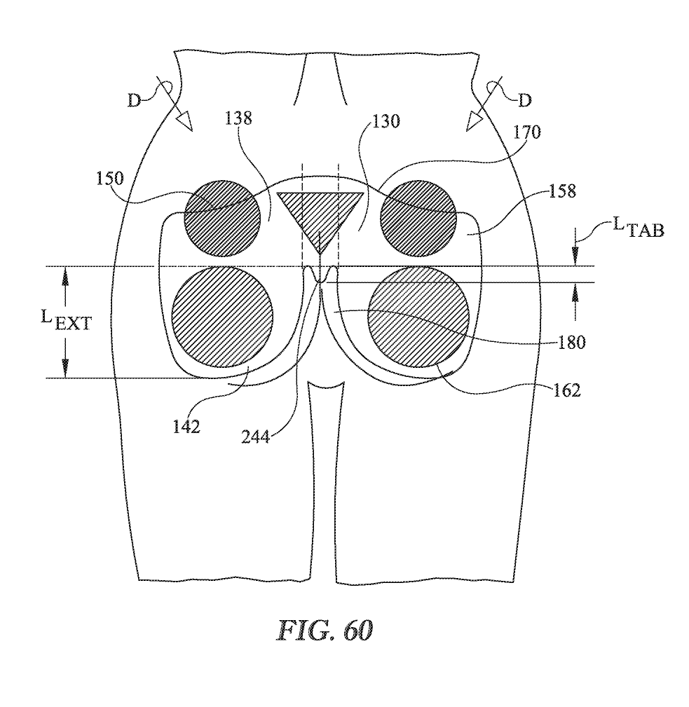

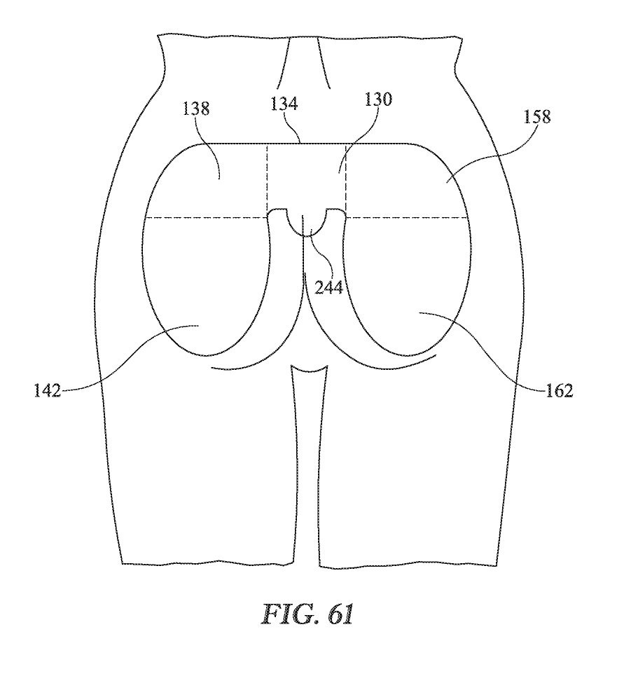

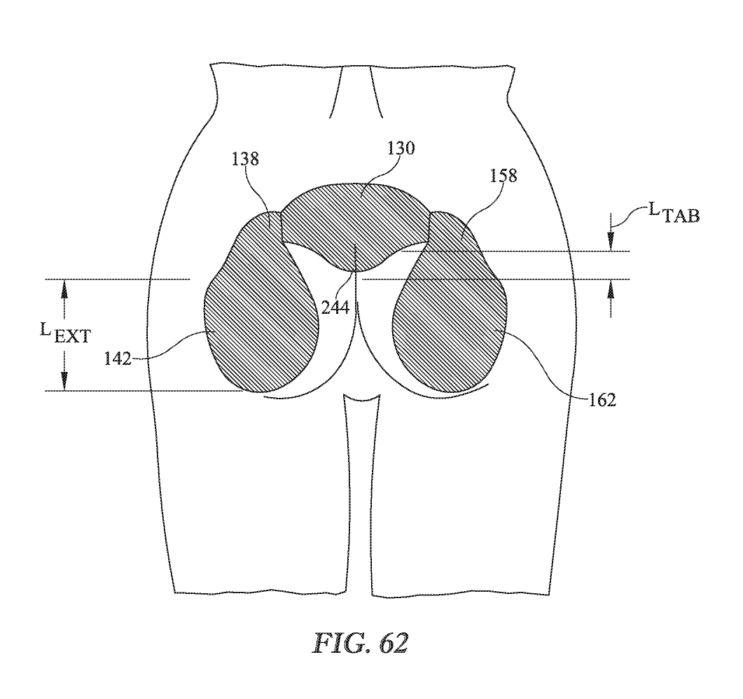

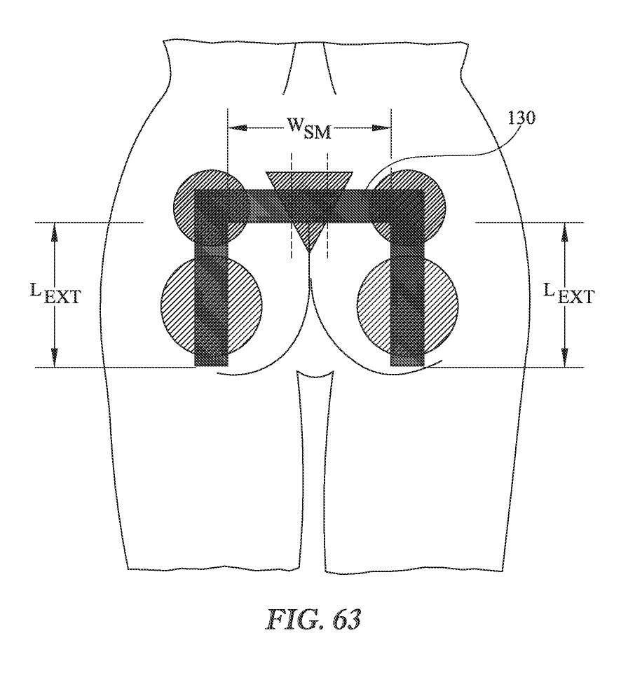

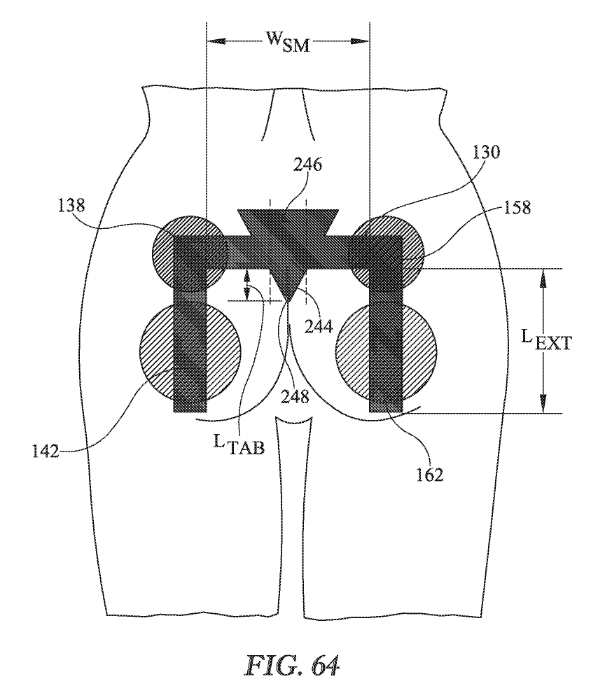

FIGS. 60-64 illustrate variations on the planform of the dressing. The planform of FIG. 60 is similar to that of FIGS. 1, 3A and 4 except that edges 150, 170 of the gluteal bases are concave rather than convex from the vantage point of an observer looking in direction D. In addition, the dressing of FIG. 60 includes a tab 244 which extends inferiorly from the inferior edge 132 of sacral member 130 at a location between the gluteal members 136, 156. Alternatively the tab may be thought of as a feature that defines the inferior edge and projects longitudinally into intermember space 180. The tab has a longitudinal dimension L_{TAB} which is less than the longitudinal dimension L_{EXT} of the gluteal extensions. In particular the tab is sized and shaped so that when the dressing is applied to a care recipient the tab conforms to and nests in the care recipient's intergluteal cleft. This helps ensure a tight seal which, in turn, helps ensure that the occlusive nature of the zone of occlusion is not compromised. The dressing planform of FIG. 61 is linear along the superior edge 134 of sacral member 130. The dressing illustrated in FIG. 62 is more irregularly shaped. The dressing illustrated in FIG. 63 has straight line borders which meet at right angles. In addition sacral member 130 is more laterally elongated than is the case in other embodiments previously described. The dressing illustrated in FIG. 64 is similar to that of FIG. 63 except at least part of the sacral member has a shape which approximates the shape of a triangle. The triangle has a base 246 corresponding approximately to the care recipient's sacral base and an apex 248. The base is superior of the apex.

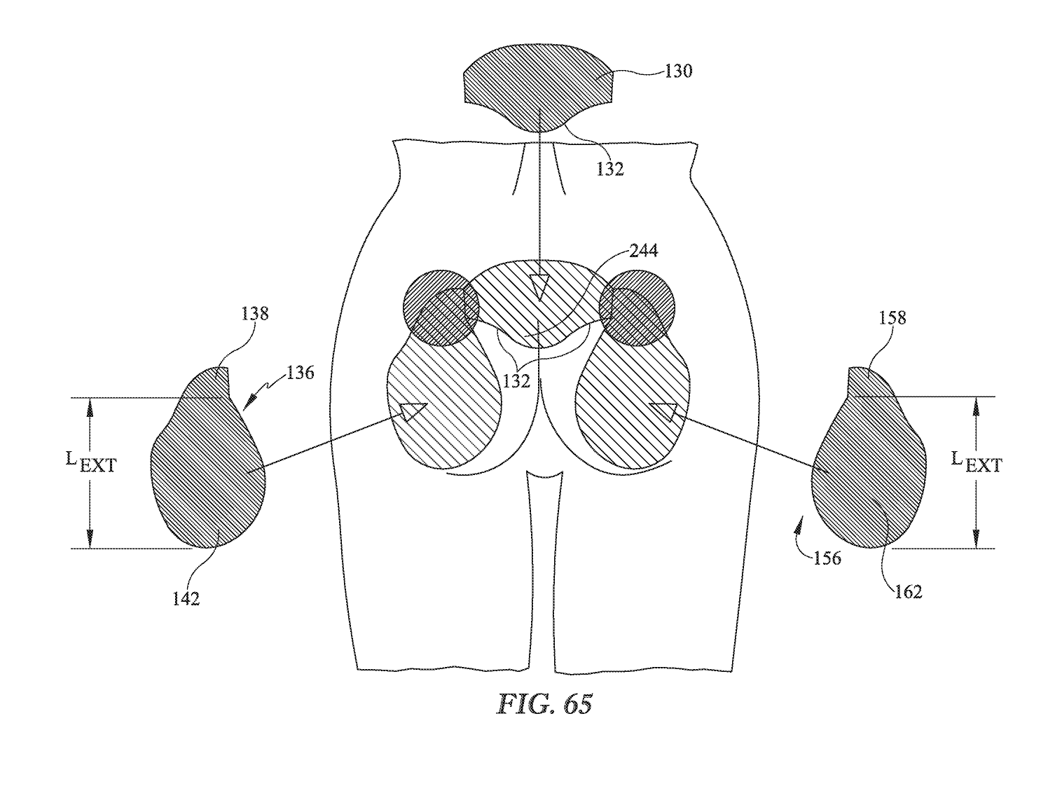

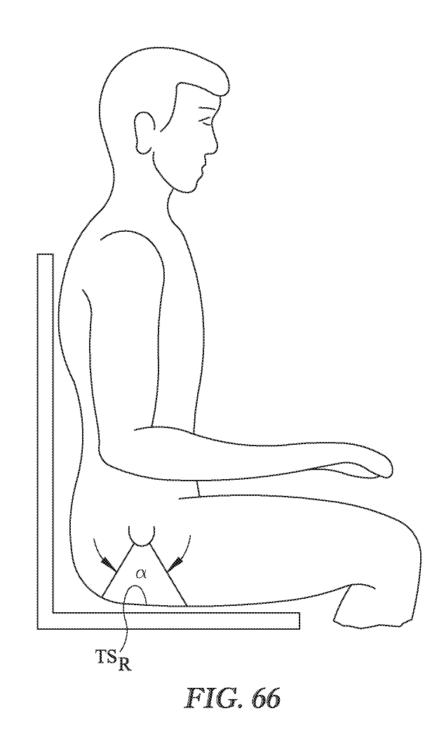

In the arrangement of FIG. 65 sacral member 130, left gluteal member 136 and right gluteal member 156 are individual members rather than being integrated into a single unit. The left and right gluteal members are dimensioned such that gluteal extensions 142, 162 will extend inferiorly of the inferior edge 132 of the sacral member when the gluteal members are applied to a care recipient such that the gluteal members border the sacral member and the sacral member is longitudinally aligned with the care recipient's sacrum.

Although the dressing has been defined in geometric terms, it can also be described in relation to a target anatomy. In practice the dressing may be sized and proportioned for a target anatomy representative of a selected population for example a 50th percentile United States male, with the understanding that the dressing may prove to be suboptimally sized for other individuals, particularly an individual whose anatomy differs markedly from the target anatomy, for example a 1st or 99th percentile United States male. Alternatively the dressing may be sized and proportioned for an extreme target anatomy such as the 99th (or 1st) percentile anatomy, with the understanding that an oversized (or undersized) dressing may nevertheless work well for at least some range of lower (or higher) percentile individuals.

Referring again to FIGS. 1-2 and 3C the dressing may be sized and proportioned according to the criteria set forth in table 1 below. In the table, W_{S, IT} is the lateral dimension of the intermember space 180 taken at a longitudinal position corresponding to the longitudinal position of the ITs of a target anatomy when the dressing is applied to the anatomy with its superior edge 134 approximately longitudinally aligned with the sacral base of the anatomy. L_{GM} is the longitudinal dimension of the left and right gluteal members:

TABLE 1

| dimension | value |
|---|---|
| W_{S, IT} | less than distance W_{IT} between left and right ischial |
| | tuberosity of the target anatomy. |
| L_{GM} | greater than or equal to a longitudinal distance L_{L5S1-IT} |
| | from the ischial tuberosity to the L5/S1 interface of the |
| | target anatomy. |

The distance W_{S, IT} in the table ensures that gluteal extensions 142, 162 are wide enough that their inner edges 144, 164 are closer to the care recipient's saggital plane P_{SAG} than the ITs are. As a result the gluteal extensions will extend laterally far enough toward the saggital plane to overlie the ITs. The distance L_{GM} ensures that the dressing extends longitudinally far enough to provide protection to the care recipient's sacral region and to the region susceptible to pressure ulcers in the vicinity of the ITs.

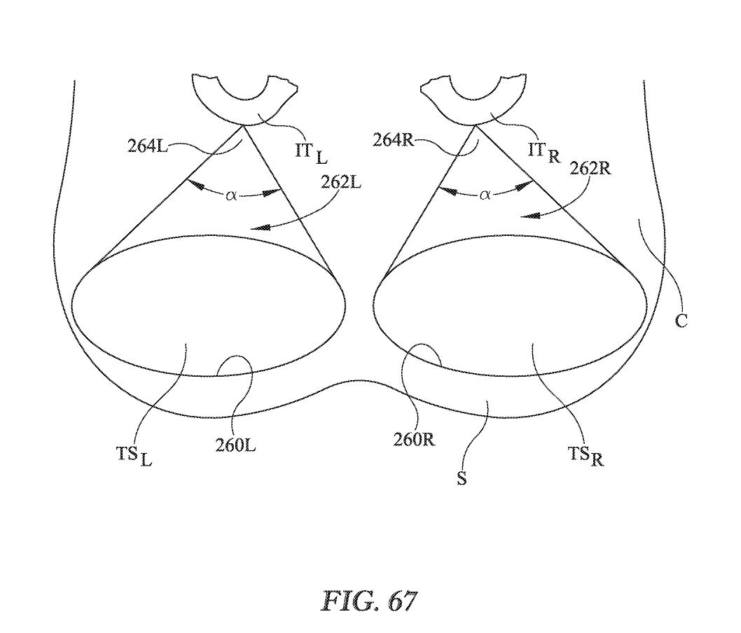

Referring additionally to FIGS. 66-67, according to another set of criteria the gluteal extensions are sized so that when the dressing is applied to a care recipient having a target anatomy with the superior edge 134 of sacral member 130 approximately longitudinally aligned with the care recipient's sacral base, the left and right gluteal extensions overlie respective left and right threat-susceptible regions TS_{L}, TS_{R} of the target anatomy. The left threat-susceptible region TS_{L} is a region bounded by the intersection 260L, when the target anatomy is in a seated posture, of the care recipient's skin S and a left notional cone 262L having a vertex 264L at the care recipient's left ischeal tuberosity IT_{L} and an opening angle α of about 90 degrees. The right threat-susceptible region TS_{R} is a region bounded by the intersection 260R, when the target anatomy is in a seated posture, of the care recipient's skin S and a right notional cone 262R whose vertex is at the care recipient's right ischeal tuberosity IT_{R} and which has an opening angle α of about 90 degrees.

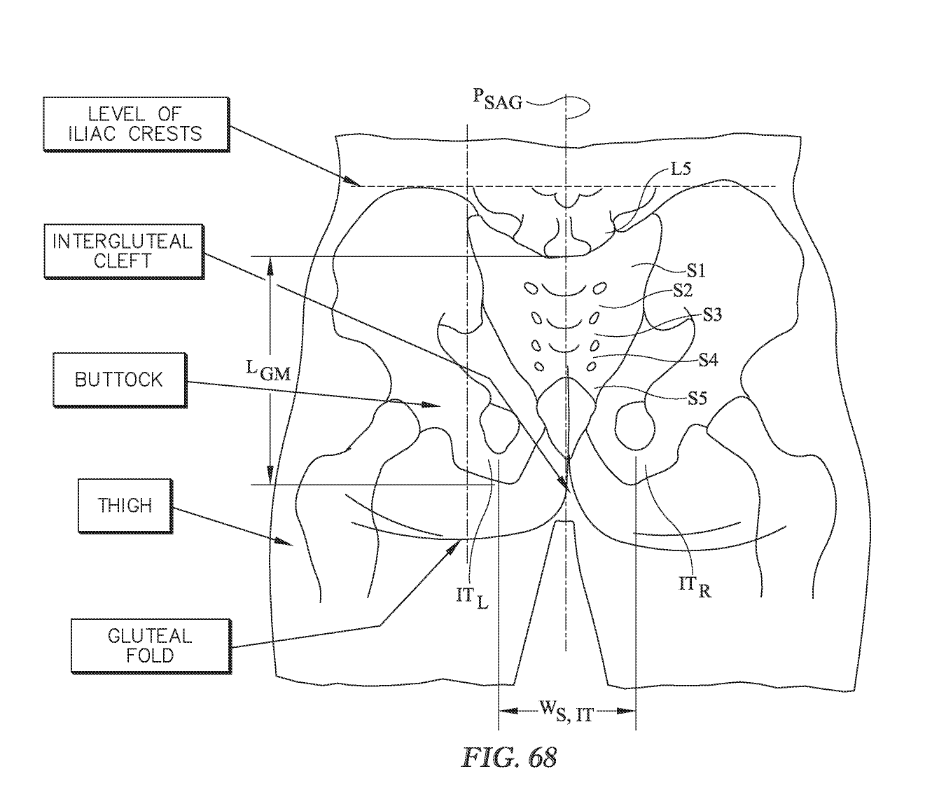

Referring to FIG. 68, according to another set of criteria the dressing is configured for a target adult care recipient according to the criteria set forth in table 2 below:

TABLE 2

| Dimension: | Value: |
|---|---|
| gluteal member | sufficiently long to extend longitudinally from the |
| longitudinal length | sacral base to the ischeal tuberosity. |
| L_{GM} | |
| gluteal member | laterally narrow enough that when each gluteal |
| intermember | member is approximately laterally centered on a |
| distance W_{S,IT} | corresponding gluteus maximus of the care recipient, |
| | inner edges 144, 164 of the gluteal members are, at |
| | a longitudinal position corresponding to the |
| | longitudinal position of the ITs, at least as laterally |
| | close to the saggital plane as the corresponding |
| | ischeal tuberosities are. |

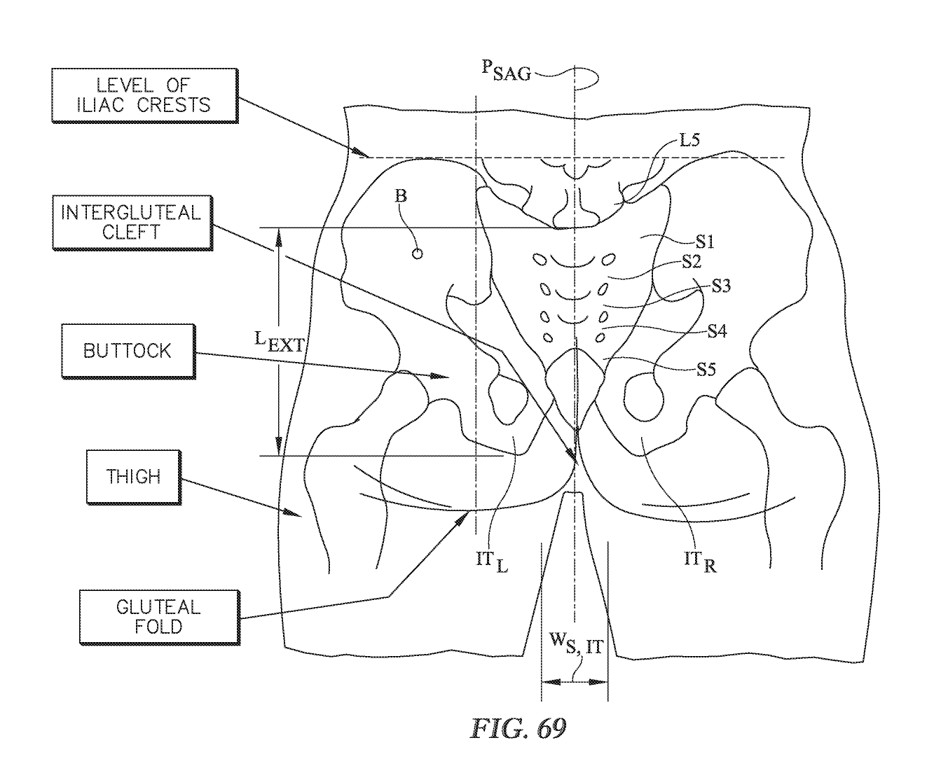

Referring to FIG. 69, according to another set of criteria the dressing is configured for a representative adult care recipient according to the criteria set forth in table 3 below:

TABLE 3

| Dimension: | Value: |
|---|---|
| longitudinal length L_{EXT} | sufficiently long to extend longitudinally |
| of the left and right | from the lateral middle “B” of the superior |
| gluteal extensions | edge of the respective left or right gluteus |
| | maximus to the ischeal tuberosity. |
| gluteal member | laterally narrow enough that when each gluteal |
| intermember | member is approximately laterally centered on |
| distance W_{S,IT} | a corresponding gluteus maximus of the care |
| | recipient, inner edges 144, 164 of the gluteal |
| | members are, at a longitudinal position |
| | corresponding to the longitudinal position |
| | of the ITs, at least as laterally close to the |
| | saggital plane as the corresponding |
| | ischeal tuberosities are. |

In yet another embodiment the preventive dressing includes a moisture barrier layer 110 and a connective element 120 on the recipient side of the dressing. As in the previously described embodiments the connective element is arranged to provide a zone of occlusion Z. The moisture barrier layer includes a sacral member 130 and a left gluteal member 136. The left gluteal member has a left base 138 which borders a left side of the sacral member and a left extension 142 which extends longitudinally inferior of the left base. The moisture barrier layer also includes a right gluteal member 156. The right gluteal member has a right base 158 which borders a right side of the sacral member and a right extension 162 which extends longitudinally inferior of the right base. The sacral member is shaped and dimensioned to overlie the sacrum of a target care recipient. The gluteal members are shaped and dimensioned to overlie the gluteus maximus muscles of the care recipient and to extend longitudinally inferiorly at least as far as the ischeal tuberosities of the care recipient when the dressing is applied to the care recipient with the sacral member approximately laterally and longitudinally aligned with the care recipient's sacrum. The dressing may also include a tab 244 which extends inferiorly from an inferior edge of the sacral member at a location between the gluteal members. The tab is sized and shaped to conform to the intergluteal cleft of the care recipient.

Although the anatomically based criteria set forth above have, in some cases, been presented separately from the geometric criteria, they do not necessarily conflict with each other. Therefore a designer may find it useful to choose design criteria from among the separately enumerated criteria.

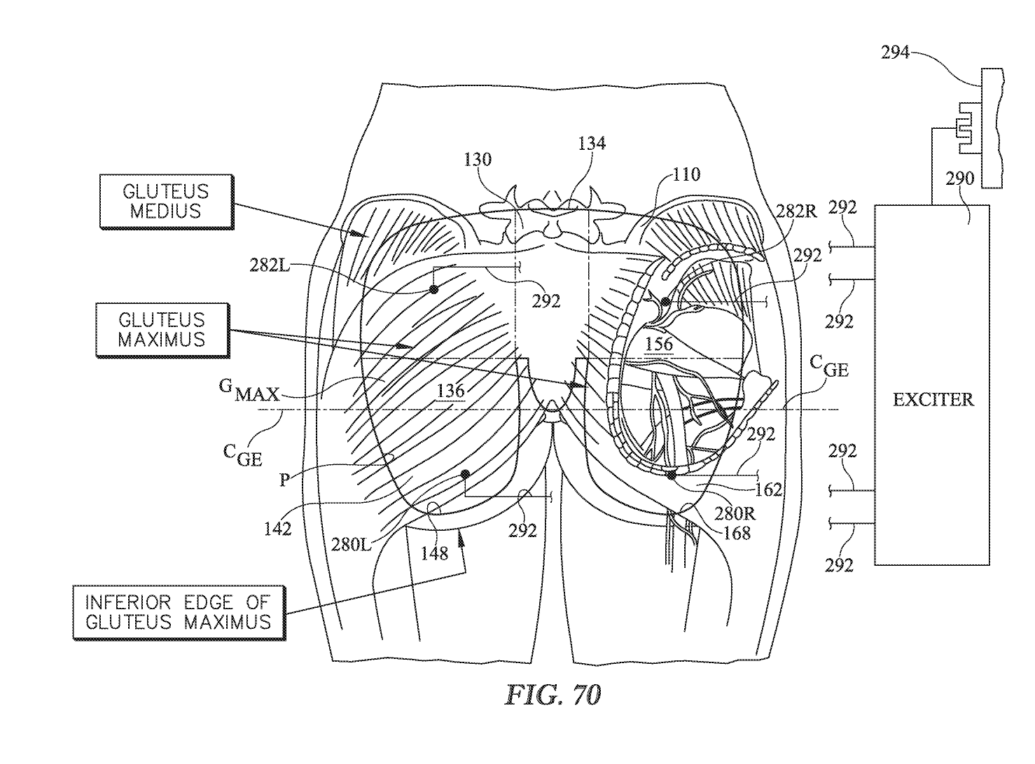

FIG. 70 shows a preventive dressing assembly 270 which comprises a dressing 100 and an exciter 280. The dressing of the dressing assembly, like the dressings already described, has a moisture barrier layer 110 with a recipient side, an environmental side, a superior edge 134, an inferior edge defined by gluteal extension inferior edges 148, 168, a perimeter P, and a connective element, such as an adhesive, on the recipient side of the moisture barrier layer.

Unlike the dressings already described the dressing 100 of the dressing assembly 270 also includes an array of electrodes. The electrode array includes a left inferior electrode 280L, a left intermediate electrode 282L, a right inferior electrode 280R, and a right intermediate electrode 282R. The intermediate electrodes are superior of the inferior electrodes. In the illustrated embodiment the electrodes are positioned relative to each other within dressing perimeter P so that the left electrodes overlie the left gluteus maximus of a care recipient and the right electrodes overlie the right gluteus maximus of the care recipient when the dressing is applied to the care recipient with sacral member 130 overlying the care recipient's sacrum. In the illustrated embodiment the intermediate electrodes 282 are longitudinally aligned with sacral member 130, and the inferior electrodes 280 are positioned near the termini 148, 168 of the gluteal extensions, for example inferior of a laterally extending centerlines C_{GE} of the gluteal members but nevertheless not where they would be inferior of the inferior edge of the gluteus maximus of the care recipient.

The dressing assembly also includes an exciter 290 connected to the electrodes by wires 292. The illustrated exciter is connected to an AC power source 294 such as a conventional outlet. Alternatively, power source 294 may be an RF power source or a near field power source. The exciter is adapted to selectively apply an intermittent excitation to specific electrodes, for example a left excitation to only the left electrodes, a right excitation to only the right electrodes, concurrent excitation to both the left electrodes and the right electrodes, and excitation to longitudinally aligned electrodes for example to the left and right intermediate electrodes. An example excitation is the application of a voltage difference across two electrodes.

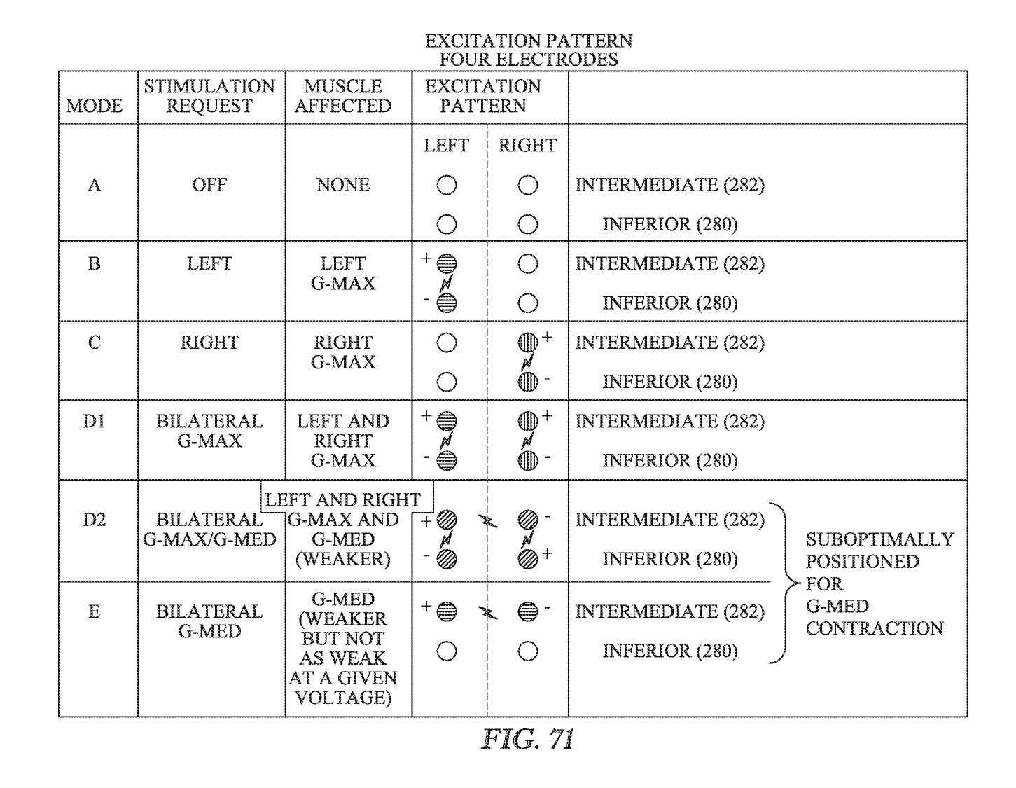

In the illustrated embodiment the left intermediate electrode 282L is laterally aligned with the left gluteal member 136 and the right intermediate electrode 282R is laterally aligned with the right gluteal member 156. The left inferior electrode 280L is on the left gluteal extension 142 longitudinally inferior of the left intermediate electrode, and the right inferior electrode 280R is on the right gluteal extension 162 inferior of the right intermediate electrode. Referring additionally to FIG. 71, exciter 290 is capable of selectively applying an excitation in each excitation mode set forth in table 4 below. A lightning bolt symbol in FIG. 71 indicates which electrodes are companions of each other for each mode of excitation. Plus and minus signs next to the electrode symbols show voltage polarity (plus for higher voltage; minus for lower voltage) for each companion pair.

TABLE 4

| Mode | Involved Electrodes | Relative Polarity |
|---|---|---|
| A | none | not applicable |
| B | left intermediate/left inferior | either |
| C | right intermediate/right inferior | either |
| D1 | left intermediate/left inferior and | same left and right |
| | right intermediate/right inferior | |
| D2 | left intermediate/left inferior and | opposite left and right |
| | right intermediate/right inferior | |
| E | left intermediate/right intermediate | either |

The dressing is sized and the electrodes are positioned so that when the dressing is applied to a care recipient having a target anatomy with each gluteal member approximately laterally centered on a corresponding gluteus maximus of the care recipient, and the sacral member approximately laterally and longitudinally aligned with the sacrum of the care recipient, the excitation stimulates one or more muscles of the care recipient as set forth in table 5 below and in FIG. 71.

TABLE 5

| | | | Primary | Secondary |
|---|---|---|---|---|
| | Involved | Relative | Muscle | Muscle |
| Mode | Electrodes | Polarity | Stimulated | Stimulated |
| A | none | not applicable | none | none |
| B | left | either | Left gluteus | |
| | intermediate/ | | maximus | |
| | left inferior | | | |
| C | right | either | Right gluteus | |
| | intermediate/ | | maximus | |
| | right inferior | | | |
| D1 | left | same left and | Left gluteus | |
| | intermediate/ | right | maximus and | |
| | left inferior | | right gluteus | |
| | and right | | maximus | |
| | intermediate/ | | | |
| | right inferior | | | |
| D2 | left | opposite left | Left gluteus | superior |
| | intermediate/ | and right | maximus and | region of |
| | left inferior | | right gluteus | left and |
| | and right | | maximus. | right gluteus |
| | intermediate/ | | | medius |
| | right inferior | | | (weak) |
| E | left | either | superior | |
| | intermediate/ | | region of | |
| | right | | left and | |
| | intermediate | | right gluteus | |
| | | | medius (not | |
| | | | as weak as | |
| | | | mode D2 | |
| | | | for a given | |
| | | | voltage) | |

Modes D1 and D2 are the same except for the voltage polarity. In mode D1 electrical current flows through the gluteal muscle tissue between left intermediate electrode 282L and left inferior electrode 280L thereby stimulating the left gluteus maximus of the care recipient. Similarly, electrical current flows through the gluteal muscle tissue between right intermediate electrode 182R and right inferior electrode 280R thereby stimulating the right gluteus maximus. The excitation causes the muscle to contract which makes the tissue less susceptible to deep tissue injury. Because the voltage at both intermediate electrodes is the higher voltage, there is no noteworthy current flow laterally between the left and right intermediate electrodes.

In mode D2 electrical current flows longitudinally between left intermediate electrode 282L and left inferior electrode 280L by way of the left gluteal muscle tissue thereby stimulating the left gluteus maximus. Electrical current flows longitudinally between right intermediate electrode 282R and right inferior electrode 280R by way of the right gluteal muscle tissue thereby stimulating the right gluteus maximus. In addition, the voltage difference between the left and right intermediate electrodes is expected to cause current to flow laterally between the left gluteus medius and the right gluteus medius thereby stimulating the gluteus medius muscles. The lateral current flow is expected to be relatively weak in comparison to the longitudinal current flow because the gluteus maximus muscles lie transversely between the intermediate electrodes and the gluteus medius muscles. The electrodes are therefore not optimally positioned to cause lateral current flow.

In mode E electrical current flows between the left and right intermediate electrodes thereby stimulating the left and right gluteus medius muscles. As with mode D2 the electrodes are not optimally positioned to stimulate the gluteus medius, however the stimulation is expected to be less weak than that of mode D2.

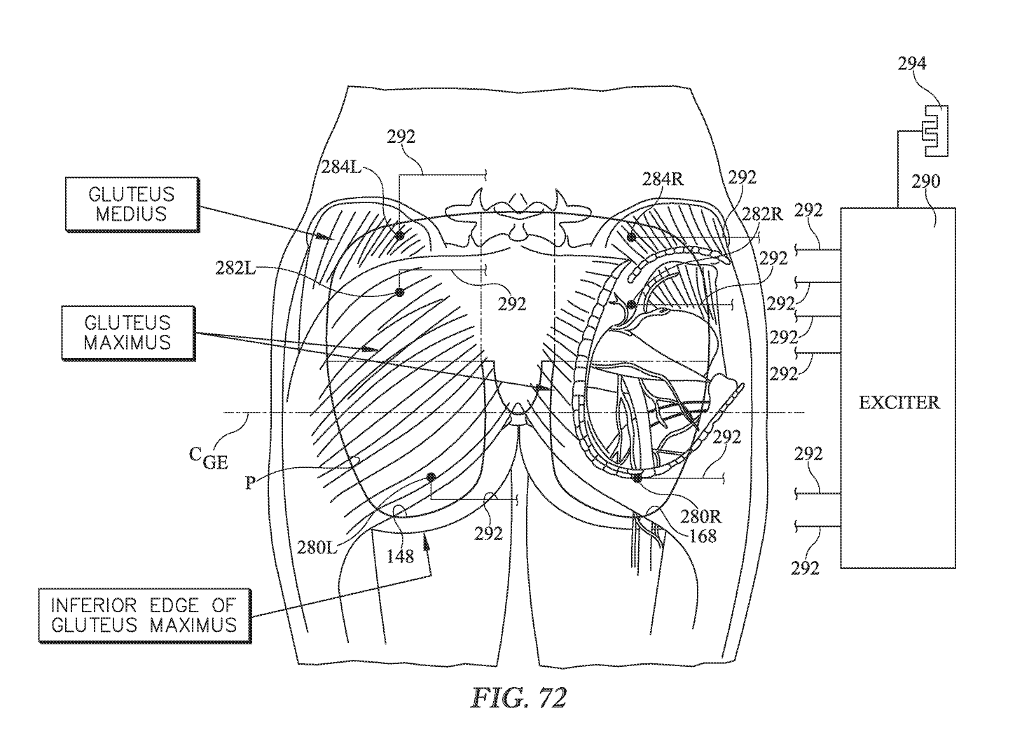

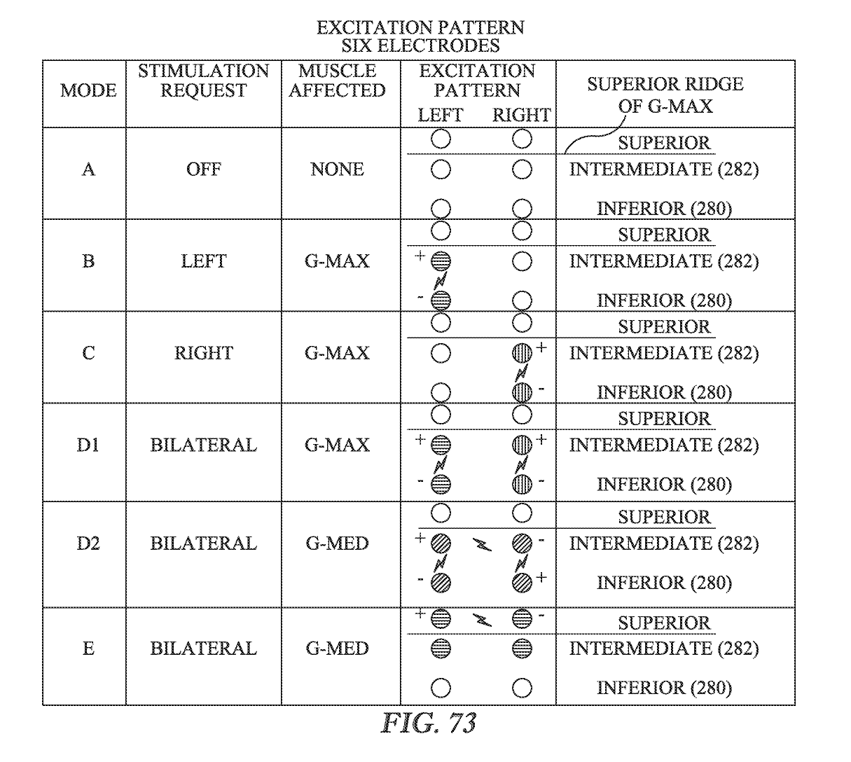

The foregoing describes a four electrode embodiment. FIGS. 72-73 shows a six electrode embodiment. In the diagrams of FIG. 73 a horizontal line schematically signifies the superior edge of the gluteus maximus and therefore distinguishes between the electrodes that are superior of and inferior of the superior edge of the gluteus maximus. The six electrode embodiment includes a left superior electrode 284L longitudinally superior of left intermediate electrode 282L and a right superior electrode 284R longitudinally superior of the right intermediate electrode 282R. In the illustrated embodiment the electrodes are positioned relative to each other within dressing perimeter P, so that the left and right superior electrodes overlie the left and right gluteus medius muscles of a care recipient when the dressing is applied to the care recipient with sacral member 130 overlying the care recipient's sacrum. In the illustrated embodiment the superior electrodes 284 are longitudinally aligned with sacral member 130, and the inferior electrodes 280 are positioned near the termini of the gluteal extensions, for example inferior of laterally extending centerlines C_{GE} of the gluteal members but nevertheless not where they would be inferior of the edge of the gluteus maximus of the care recipient.

The dressing assembly also includes an exciter 290 connected to the electrodes by wires 292. The illustrated exciter is connected to an AC power source 294 such as a conventional outlet. Alternatively, power source 294 may be an RF power source or a near field power source. The exciter is adapted to selectively apply an excitation to specific electrodes, for example a left excitation to only the left electrodes, a right excitation to only the right electrodes, concurrent excitation to both the left electrodes and the right electrodes, and excitation to longitudinally aligned electrodes for example to the left and right superior electrodes. An example excitation is the application of a voltage difference across two electrodes.

In the illustrated embodiment the left superior electrode 284L is laterally aligned with left gluteal member 136 and the right superior electrode 284R is laterally aligned with the right gluteal member 156. The left inferior electrode 280L is on the left gluteal extension 142 longitudinally inferior of the left intermediate electrode 282L, and the right inferior electrode 280R is on the right gluteal extension 162 longitudinally inferior of the right intermediate electrode. Exciter 290 is capable of selectively applying an excitation in each excitation mode set forth in table 7 below. A lightning bolt symbol in FIG. 73 indicates which electrodes are companions of each other for each mode of excitation. Plus and minus signs next to the electrode symbols show voltage polarity (plus for higher voltage; minus for lower voltage) for each companion pair.

TABLE 7

| Mode | Involved Electrodes | Relative Polarity |
|---|---|---|
| A | none | either |
| B | left intermediate/left inferior | either |
| C | right intermediate/right inferior | either |
| D1 | left intermediate/left inferior and | same left and right |
| | right intermediate/right inferior | |
| D2 | left intermediate/left inferior and | opposite left and right |
| | right intermediate/right inferior | |
| E | left superior/right superior | either |

The dressing is sized and the electrodes are positioned so that when the dressing is applied to a care recipient having a target anatomy with each gluteal member approximately laterally centered on a corresponding gluteus maximus of the care recipient, and the sacral member approximately laterally and longitudinally aligned with the sacrum of the care recipient, the excitation stimulates one or more muscles of the care recipient as set forth in table 8 below and in FIG. 73.

TABLE 8

| | | | Primary | Secondary |
|---|---|---|---|---|
| | Involved | Relative | Muscle | Muscle |
| Mode | Electrodes | Polarity | Stimulated | Stimulated |
| A | none | not | none | none |
| | | applicable | | |
| B | left intermediate/ | either | Left gluteus | |
| | left inferior | | maximus | |
| C | right | either | Right gluteus | |
| | intermediate/ | | maximus | |
| | right inferior | | | |
| D1 | left intermediate/ | same left and | Left gluteus | |
| | left inferior | right | maximus and | |
| | and right | | right gluteus | |
| | intermediate/ | | maximus | |
| | right inferior | | | |
| D2 | left intermediate/ | opposite left | Left gluteus | superior |
| | left inferior | and right | maximus and | region of |
| | and right | | right gluteus | left and |
| | intermediate/ | | maximus | right gluteus |
| | right inferior | | | medius |
| | | | | (weak) |
| E | left superior/ | either | superior | |
| | right superior | | region of | |
| | | | left and right | |
| | | | gluteus | |
| | | | medius | |

Modes D1 and D2 are the same except for the voltage polarity. In mode D1 electrical current flows between the left intermediate electrode 282L and the left inferior electrode 280L by way of the left gluteus maximus thereby stimulating the left gluteus maximus. Similarly, electrical current flows from right intermediate electrode 282R to the right inferior electrode 280R by way of the right gluteus maximus thereby stimulating the right gluteus maximus. The excitation causes the gluteus maximus muscles to contract which makes the tissue less susceptible to deep tissue injury. Because the voltage at both intermediate electrodes is the higher voltage, there is no noteworthy current flow laterally between the left and right intermediate electrodes.

In mode D2 electrical current flows through the left gluteus maximus between left intermediate electrode 282L and left inferior electrode 280L thereby stimulating the left gluteus maximus. Electrical current flows through the right gluteus maximus between the right intermediate electrode 282R and the right inferior electrode 280R thereby stimulating the right gluteus maximus. In addition, the voltage difference between the left and right intermediate electrodes is expected to cause current to flow laterally between the left gluteus medius and the right gluteus medius thereby stimulating the gluteus medius muscles. The lateral current flow is expected to be relatively weak in comparison to the longitudinal current flow because the gluteus maximus muscles lie transversely between the intermediate electrodes and the gluteus medius muscles. The electrodes are therefore not optimally positioned to cause lateral current flow.

In mode E electrical current flows between the left and right superior electrodes thereby stimulating the left and right gluteus medius muscles.

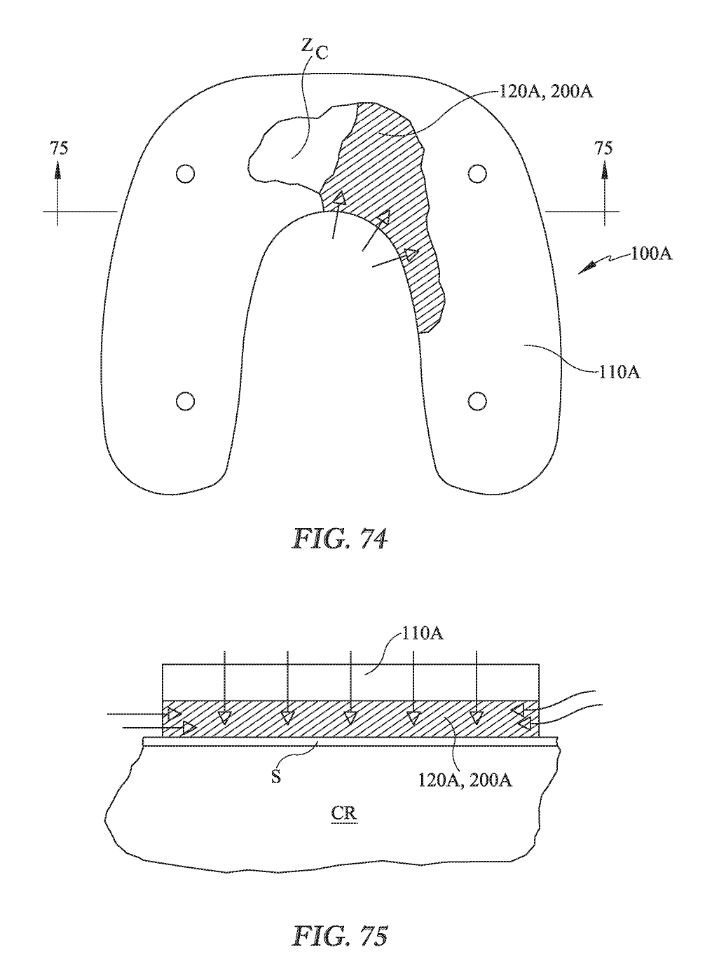

In yet another set of embodiments the electrode arrangements and electrode excitation patterns are as already described in connection with FIGS. 70-73, however other elements of the dressing do not cooperate to provide a zone of occlusion. Referring to FIGS. 74-75, a dressing 100A includes an external layer 110A which is analogous to moisture barrier layer 110 as previously described but which does not prevent moisture transfer from the environment to the portion of the care recipient's skin covered by the dressing. In addition, connective element 120A (illustrated as a spatially distributed adhesive 200A) is an adhesive element which also does not protect against moisture ingress. As a result, the region of the care recipient's anatomy covered by the dressing is a zone of coverage Z_{C} rather than a zone of occlusion Z. In such an embodiment the dressing serves as an electrode host which is configured to place the electrodes at the desired location on the anatomy of the care recipient, but does not protect against moisture ingress into the area covered by the dressing (i.e. into zone Z_{C}). This is indicated by the solid arrows which penetrate transversely through external layer 110A and connective element 120A and which also penetrate longitudinally and laterally into connective element 120A. FIG. 74 shows the four electrode variant already described in connection with FIGS. 70-71, however dressing 100A can also accommodate the six electrode variant of FIGS. 72-73.

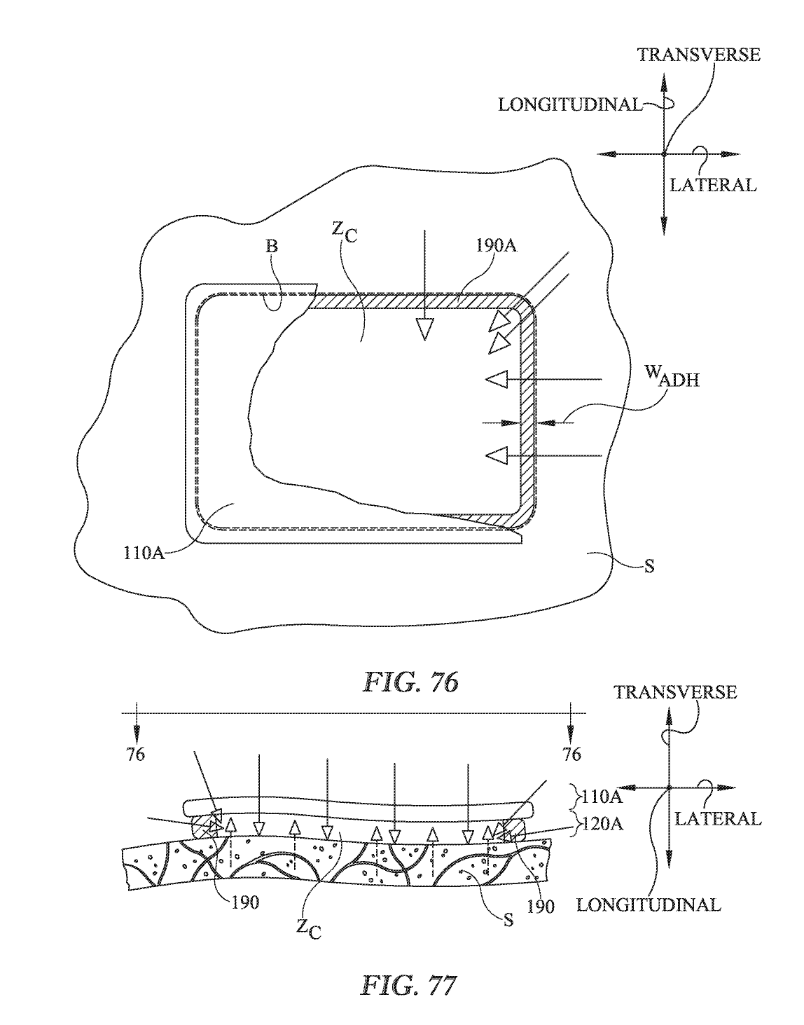

FIGS. 76-77 show an embodiment similar to that of FIGS. 74-75 but in which the connective element 120A is illustrated as an adhesive strip 190A. Moisture transfer is indicated by the solid arrows which penetrate transversely through external layer 110A and which also penetrate longitudinally and laterally through adhesive strip 190A. Once again the dressing serves as an electrode host which is configured to place the electrodes at the desired location on the anatomy of the care recipient but which does not protect against moisture ingress transversely across external layer 110A or laterally and longitudinally through adhesive strip 190A.

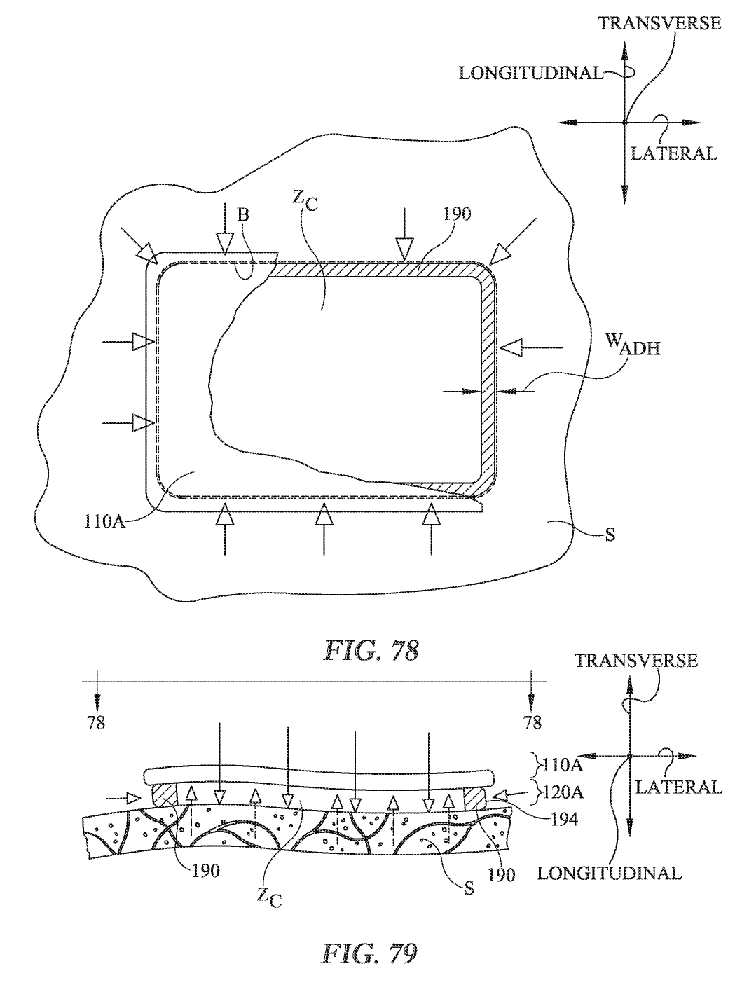

FIGS. 78-79 show an embodiment similar to that of FIGS. 76-77 but in which the connective element 120 is a an adhesive strip 190 which blocks moisture transfer laterally and longitudinally into zone Z_{C}. Moisture can nevertheless enter zone Z_{C} by way of external layer 110A. This is indicated by the solid arrows which penetrate transversely through external layer 110A and by the laterally and longitudinally directed solid arrows which terminate without crossing adhesive strip 190. Once again the dressing serves as an electrode host which is configured to place the electrodes at the desired location on the anatomy of the care recipient but which does not protect against moisture ingress transversely across external layer 110A.

The variations in planform, such as those of FIGS. 60-65, apply equally to the non-occlusive embodiments just described. In addition a pressure equalizing layer such as layer 220 described in connection with FIGS. 43-50 may also be incorporated.

Although this disclosure refers to specific embodiments, it will be understood by those skilled in the art that various changes in form and detail may be made without departing from the subject matter set forth in the accompanying claims.

We claim:

1. A preventive dressing having a recipient side, an environmental side, and a perimeter, the dressing comprising:
A) a moisture barrier layer having:
a) a sacral member having a longitudinal length and an inferior edge;
b) a left gluteal member having a base which borders a left side of the sacral member, and a left extension which extends longitudinally inferiorly further than the inferior edge by a left gluteal extension dimension which is approximately no less than the longitudinal length of the sacral member;
c) a right gluteal member having a base which borders a right side of the sacral member, and a right extension which extends longitudinally inferiorly further than the inferior edge by a right gluteal extension dimension which is approximately no less than the longitudinal length of the sacral member so that the left gluteal member and the right gluteal member define an intermember space; and
B) a connective element on the recipient side of the dressing, the connective element being arranged to provide a zone of occlusion.

2. The dressing of claim 1 wherein the connective element comprises an adhesive.

3. The dressing of claim 2 wherein the adhesive is one-way liquid absorbant.

4. The dressing of claim 2 wherein the adhesive is one-way liquid permeable.

5. The dressing of claim 1 wherein the connective element is a continuous strip of adhesive extending along substantially the perimeter of the dressing.

6. The dressing of claim 5 wherein the adhesive strip borders the occlusion zone and wherein an auxiliary layer resides in the occlusion zone, and the auxiliary layer comprises at least one of:
a liquid absorbant layer,
a liquid permeable layer,
a pressure equalizing layer.

7. The dressing of claim 1 wherein the connective element comprises a sacral adhesive layer and a gluteal adhesive element and wherein:
the sacral adhesive layer is an adhesive which forms substantially all of:
a) the recipient side of the sacral member, and
b) the recipient sides of the left and right gluteal bases,
the gluteal extensions have a margin and the gluteal adhesive element is a continuous strip of adhesive extending along substantially the margin of the gluteal member extensions.

8. The dressing of claim 7 including at least one auxiliary layer residing in the zone of occlusion.

9. The dressing of claim 8 wherein the auxiliary layer comprises at least one of:
A) a liquid absorbant layer,
B) a liquid permeable layer, and
C) a pressure equalizing layer.

10. The dressing of claim 1 wherein the connective element is an adhesive which forms substantially all of the recipient side of the dressing.

11. The dressing of claim 10 including at least one auxiliary layer residing in the zone of occlusion.

12. The dressing of claim 11 wherein the auxiliary layer comprises at least one of:
A) a liquid absorbant layer,
B) a liquid permeable layer, and
C) a pressure equalizing layer.

13. The dressing of claim 10 wherein the connective element comprises a first adhesive composition forming substantially all of the recipient side of the sacral member and the gluteal bases, and a second adhesive composition forming substantially all of the recipient side of the gluteal extensions and wherein the first adhesive composition adheres relatively more strongly to human skin and the second adhesive composition adheres relatively more weakly to human skin.

14. The dressing of claim 1 including a pressure equalizing layer spanning laterally across a longitudinally extending centerline of the dressing in the zone of occlusion to define a pressure equalizing zone, the pressure equalizing layer imparting a stiffness to the pressure equalizing zone which is greater than the stiffness of the dressing laterally to the left and right of the pressure equalizing zone.

15. The dressing of claim 1 including a pressure equalizing zone spanning laterally across a longitudinally extending centerline of the dressing in the zone of occlusion, the pressure equalizing zone having a planform and a thickness which is greater than the thickness of the dressing outside the planform of the pressure equalizing zone.

16. The dressing of claim 1 including a zone spanning laterally across a longitudinally extending centerline of the dressing in the zone of occlusion, the zone having a customizable thickness.

17. The dressing of claim 16 wherein the customizable thickness of the zone of customizable thickness is attributable to peel-away sublayers.

18. The dressing of claim 1 including a classified zone spanning laterally across a longitudinally extending centerline of the dressing in the zone of occlusion, the classified zone having a class specific thickness which is one of a set of two or more class specific thicknesses.

19. The dressing of claim 14 wherein the pressure equalizing layer is confined to the sacral member.

20. The dressing of claim 1 including a moisture management layer in the zone of occlusion.

21. The dressing of claim 20 wherein the moisture management layer is a liquid absorbant layer.

22. The dressing of claim 20 wherein the moisture management layer is a liquid permeable layer.

23. The dressing of claim 1 including a moisture management layer and a pressure equalizing layer both of which are in the zone of occlusion and transversely between the moisture barrier layer and the connective element.

24. The dressing of claim 23 wherein the pressure equalizing layer is confined to the sacral member.

25. The dressing of claim 1 comprising a tab which extends inferiorly from the sacral member at a location between the gluteal members, the tab having a longitudinal dimension which is less than the longitudinal dimension of the extensions.

26. The dressing of claim 1 wherein dimensions of the dressing are as set forth in the table below in which
W_{S, IT} is a lateral dimension of the intermember space taken at a longitudinal position corresponding to the longitudinal position of the ITs of a target anatomy when the dressing is applied to the anatomy with its superior edge approximately longitudinally aligned with the sacral base of the anatomy and
L_{GM} is the longitudinal dimension of the left and right gluteal members:

| dimension | value |
|---|---|
| W_{S,IT} | less than distance between left and right ischial |
| | tuberosity of the target anatomy. |
| L_{GM} | greater than or equal to a longitudinal distance |
| | L_{L5S1-IT} from the ischial tuberosity to the |
| | L5/S1 interface of the target anatomy. |

27. The dressing of claim 26 wherein the target anatomy is that of a 50th percentile United States male.

28. The dressing of claim 1 wherein the gluteal extensions are sized so that when the dressing is applied to a care recipient having a target anatomy comprising a skin, a left ischeal tuberosity and a right ischeal tuberosity so that its superior edge is approximately longitudinally aligned with the care recipient's sacral base:
the left gluteal extension covers a left threat-susceptible region of the target anatomy, the left target region being the intersection of the skin and a left notional cone when the target anatomy is in a seated posture, the left cone having a vertex at the left ischeal tuberosity and an opening angle of about 90 degrees, and
the right gluteal extension covers a right threat-susceptible region of the target anatomy, the right target region being the intersection of the skin and a right notional cone when the target anatomy is in a seated posture, the right cone having a vertex at the right ischeal tuberosity and an opening angle of about 90 degrees.

29. The dressing of claim 1 comprising a tab which extends inferiorly from the sacral member at a location between the gluteal members, the tab being sized and shaped to conform to the intergluteal cleft of a care recipient thereby preserving occlusivity of the zone of occlusion.

30. The dressing of claim 1 wherein at least part of the sacral member has a shape which approximates the shape of a triangle having a base and an apex, the base being superior of the apex.

31. The dressing of claim 1 wherein the sacral member, the left gluteal member and the right gluteal member are individual members and wherein the left and right gluteal members are dimensioned such that the extensions thereof extend inferiorly of the inferior edge of the sacral member when the gluteal members are applied to a care recipient such that the gluteal members border the sacral member and the sacral member longitudinally aligned with the care recipient's sacrum.

32. The dressing of claim 1 wherein the dressing is configured for a target adult care recipient having a sacrum, a sacral base, a left gluteus maximus having a superior edge, a right gluteus maximus having a superior edge, a left ischeal tuberosity, and a right ischeal tuberosity as set forth below:

| Dimension: | Value: |
|---|---|
| gluteal member | sufficiently long to extend longitudinally from |
| longitudinal length | the sacral base to the ischeal tuberosity. |
| L_{GM} | |
| gluteal member | laterally narrow enough that when each gluteal |
| intermember | member is approximately laterally centered on a |
| distance W_{S,IT} | corresponding gluteus maximus of the care |
| | recipient, inner edges of the gluteal members |
| | are, at a longitudinal position corresponding to |
| | the longitudinal position of the ITs, at least as |
| | laterally close to the saggital plane as the |
| | corresponding ischeal tuberosities are. |

33. The dressing of claim 1 wherein the dressing is configured for a target adult care recipient having a sacrum, a sacral base, a left gluteus maximus, a right gluteus maximus, a left ischeal tuberosity, and a right ischeal tuberosity as set forth below:

| Dimension: | Value: |
|---|---|
| gluteal extension | sufficiently long to extend longitudinally from |
| longitudinal length | the lateral middle of the superior edge of the |
| L_{EXT} | gluteus maximus to the ischeal tuberosity. |
| gluteal member | laterally narrow enough that when each gluteal |
| intermember | member is approximately laterally centered on a |
| distance W_{S,IT} | corresponding gluteus maximus of the care |
| | recipient, inner edges of the gluteal members |
| | are, at a longitudinal position corresponding to |
| | the longitudinal position of the ITs, at least as |
| | laterally close to the saggital plane as the |
| | corresponding ischeal tuberosities are. |

34-56. (canceled)
